SEQUENCE LISTING (22 sequences)

SEQ ID NO: 1 - DNA, 40 bp, Artificial Sequence
  synthetic oligonucleotide
cgaccgatgaatagcggtcagatccgtacctactcgtagc

SEQ ID NO: 2 - DNA, 36 bp, Artificial Sequence
  synthetic oligonucleotide
cgagtaggtacggatctgcgtattgcgaacgactcg

SEQ ID NO: 3 - DNA, 36 bp, Artificial Sequence
  synthetic oligonucleotide
cgagtcgttcgcaatacggctgtacgtatggtctcg

SEQ ID NO: 4 - DNA, 36 bp, Artificial Sequence
  synthetic oligonucleotide
cgagaccatacgtacagcaccgctattcatcggtcg

SEQ ID NO: 5 - DNA, 40 bp, Artificial Sequence; identical to SEQ ID NO: 1
  synthetic oligonucleotide

SEQ ID NO: 6 - DNA, 40 bp, Artificial Sequence
  synthetic oligonucleotide
cgagtaggtacggatctgcgtattgcgaacgactcgtagc

SEQ ID NO: 7 - DNA, 36 bp, Artificial Sequence; identical to SEQ ID NO: 3
  synthetic oligonucleotide

SEQ ID NO: 8 - DNA, 36 bp, Artificial Sequence; identical to SEQ ID NO: 4
  synthetic oligonucleotide

SEQ ID NO: 9 - DNA, 40 bp, Artificial Sequence; identical to SEQ ID NO: 1
  synthetic oligonucleotide

SEQ ID NO: 10 - DNA, 40 bp, Artificial Sequence; identical to SEQ ID NO: 6
  synthetic oligonucleotide

SEQ ID NO: 11 - DNA, 40 bp, Artificial Sequence
  synthetic oligonucleotide
cgagtcgttcgcaatacggctgtacgtatggtctcgtagc

SEQ ID NO: 12 - DNA, 36 bp, Artificial Sequence; identical to SEQ ID NO: 4
  synthetic oligonucleotide

SEQ ID NO: 13 - DNA, 40 bp, Artificial Sequence; identical to SEQ ID NO: 1
  synthetic oligonucleotide

SEQ ID NO: 14 - DNA, 40 bp, Artificial Sequence; identical to SEQ ID NO: 6
  synthetic oligonucleotide

SEQ ID NO: 15 - DNA, 40 bp, Artificial Sequence; identical to SEQ ID NO: 11
  synthetic oligonucleotide

SEQ ID NO: 16 - DNA, 40 bp, Artificial Sequence
  synthetic oligonucleotide
cgagaccatacgtacagcaccgctattcatcggtcgtagc

SEQ ID NO: 17 - RNA, 21 nt, Artificial Sequence
  synthetic oligonucleotide
gugcgcugcuggugccaacuu

SEQ ID NO: 18 - DNA, 23 bp, Artificial Sequence
  synthetic oligonucleotide
guuggcaccagcagcgcacgcta

SEQ ID NO: 19 - DNA, 23 bp, Artificial Sequence
  synthetic oligonucleotide
gugcgcugcuggugccaacgcta

SEQ ID NO: 20 - RNA, 21 nt, Artificial Sequence
  synthetic oligonucleotide
guuggcaccagcagcgcacuu

SEQ ID NO: 21 - DNA, 23 bp, Artificial Sequence
  synthetic oligonucleotide
gcaagcugacccugaaguugcta

SEQ ID NO: 22 - DNA, 21 bp, Artificial Sequence
  synthetic oligonucleotide
aacuucagggucagcuugcuu

(19) United States
(12) Patent Application Publication
(10) Pub. No.: US 2010/0323018 A1
(43) Pub. Date: Dec. 23, 2010

(54) Branched DNA/RNA monomers and uses thereof

(75) Inventors: Darrell J. Irvine, Arlington, MA (US); Soong Ho Um, Gwangsan-gu (KR)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/456,592

(22) Filed: Jun. 17, 2009

(51) Int. Cl. A61K 9/14; C07H 21/02; A61K 31/7105; C12P 19/34; A61P 43/00

(52) U.S. Cl. 424/489; 536/24.5; 514/44 A; 435/91.52

(57) ABSTRACT

The invention provides compositions and methods relating to delivery of agents in vivo or in vitro. More specifically, the invention provides nanoparticles synthesized from crosslinked nucleic acids, optionally having a lipid shell or coating, and may further comprise for example small molecule or high molecular weight compounds as therapeutic or diagnostic agents.

### FEDERALLY SPONSORED RESEARCH

This invention was made with Government support under grant number DMR-0819762 from the National Science Foundation (NSF MRSEC). The Government has certain rights to this invention.

### BACKGROUND OF INVENTION

In vivo drug delivery approaches to date have focused in part on liposome-mediated delivery and biodegradable polymeric particles. Liposomes are the prototypical nanoscale drug carrier and have a variety of favorable properties, such as biocompatibility and biodegradability and an ability for sustained circulation times in the blood. However, liposomes are also known to be unstable in the presence of serum, often encapsulate only low levels of hydrophilic drugs, and have a limited ability to regulate the release of hydrophobic compounds. Biodegradable polymeric nanoparticles have been pursued as an alternative, but these synthetic particles also encapsulate relatively low levels of proteins or hydrophilic drugs and tend to have lower blood circulation times than liposomes.

Carriers for delivery of RNA-based agents have become of interest in order to modulate protein expression through RNA interference in the treatment of disease. However, the RNA used in these approaches are relatively unstable in vivo and must be delivered to the cytosol of cells, a process that has been demonstrated to be inefficient when naked RNA is used. Clinical implementation of RNA interference approaches would benefit from efficient in vivo delivery of RNA-based agents.

### SUMMARY OF INVENTION

The invention relates broadly to the delivery, including sustained delivery, of agents such as therapeutic and diagnostic agents and including RNA-based agents (e.g., siRNA agents) in vivo and in vitro. Aspects of the invention relate to branched nucleic acid monomers comprising siRNA, and hydrogels and nanoparticles made from the crosslinking of such monomers, as illustrated in FIGS. 1 and 2.

It has been unexpectedly found according to the invention that the activity of some siRNA forms can be reduced, in some instances partially and in other instances nearly completely, when bound to branched nucleic acid monomers. More specifically, L-form siRNA has been shown to have reduced interference activity when bound to a branched DNA monomer while R-form siRNA retains its gene silencing activity. The difference in interference activity between these two forms when bound to a branched nucleic acid is surprising and unexpected at least because when used in free form these forms both have significant silencing activities. As shown in the Examples, the L-form siRNA when used in free form may reduce expression of a target to a level that is about 20% of a control (i.e., expression levels in the absence of exogenously applied siRNA). However, when that L-form siRNA is attached to an X-DNA, it only reduces expression of the same target to a level that is about 60-80% of the control (i.e., only a 20-40% reduction as compared to an 80% reduction when used in free form). In contrast, attachment of R-form siRNA to X-DNA did not impact the ability of the siRNA to reduce expression to an appreciable extent. One of ordinary skill in the art would not have predicted such disparity in the L- and R-forms particularly in view of their comparable efficacy when used in free form. The finding suggests that preferential use of the R-form in the context of a branched nucleic acid for gene silencing in vitro and in vivo should provide more efficient (and higher specific activity) gene silencing, and should prevent administration or exposure to unnecessary and ineffective agents.

The invention further provides methods for producing nanoparticles comprising crosslinked versions of these branched nucleic acid/siRNA monomers. A schematic of this method is provided in FIG. 3. The nanoparticles produced according to these methods are non-toxic owing to the nucleic acid matrix at their core and to the absence of organic solvents in the synthesis process. In addition to their siRNA payload, these nanoparticles may also comprise other agents ranging from small molecules to high molecular weight compounds (such as proteins) and have been shown to provide significantly extended release profiles for both.

The invention therefore provides in part compositions and methods for efficient and non-toxic delivery of siRNA agents locally or systemically. Unlike prior art approaches, the submicron particles (also referred to herein as nanoparticles) provided by the invention comprising crosslinked branched nucleic acids complexed with siRNA monomers do not require electrostatic complexing of with a cytosolic delivery agent such as polyethyleneimine, cationic lipids, or other cell-entry reagents. The lack of a requirement for physical complexation of the RNA-based agent with a cell-entry reagent avoids the need to unpack the siRNA from the reagent, a process that has been problematic and may increase the half-life of the siRNA by avoiding the rapid clearance of cationic carriers complexed to anionic siRNA by the reticuloendothelial system, and avoids the severe toxicity observed for such carriers at least in animal models.

Thus, in one aspect the invention provides a complex comprising a branched nucleic acid, and an R-form siRNA linked (or attached, as the terms are used interchangeably herein) to a branched nucleic acid. The siRNA may be linked to an arm of the branched nucleic acid. The resulting complex will therefore comprise at least one siRNA attached to the branched nucleic acid and having a 3′ antisense overhang.

In this and other aspects of the invention, the branched nucleic acid may be an X-shaped nucleic acid, or a Y-shaped nucleic acid, or T-shaped nucleic acid, or a dendrimeric nucleic acid. The branched nucleic acid may be comprised of DNA or DNA analogs. Thus, the branched nucleic acid is a branched DNA in some embodiments.

In some embodiments, the R-form siRNA is covalently attached to the branched nucleic acid while in other embodiments it is non-covalently attached to the branched nucleic acid. Attachment may be direct or it may be indirect and thereby involve an intermediary such as a linker. The branched nucleic acid may be linked to 2, 3, 4 or more siRNA, such as 2, 3, 4, or more R-form siRNA. In some embodiments, R-form siRNA is linked to each arm of the branched nucleic acid.

In another aspect, the invention provides a method comprising contacting an R-form siRNA with a branched nucleic acid, thereby forming a complex comprising a branched nucleic acid linked to an R-form siRNA.

In another aspect, the invention provides matrix comprising any of the afore-mentioned complexes in a crosslinked form, for example as a hydrogel. Thus, the invention provides a hydrogel comprising any of the aforementioned complexes wherein the complexes are crosslinked to each other.

In another aspect, the invention provides a composition comprising a hydrogel comprising crosslinked branched DNA complexes comprising to R-form siRNA (or branched DNA/R-form siRNA complexes).

In these and other aspects of the invention, the complexes may be homogeneous or heterogenous. Thus, the complexes within a hydrogel may differ from each other with respect to their make up (e.g., some may be all DNA, some may have DNA analogs, etc.), their siRNA content (e.g., some may have 1 R-form siRNA, some may have more than 1 R-form siRNA, some may have L-form siRNA, etc.), the location or attachment points of siRNA, etc.

In some embodiments, the individual branched DNA complexes comprise 1, 2, 3 or 4 R-form siRNA. In some embodiments, the R-form siRNA is not complexed with cationic polymers or lipids. The hydrogel may lack an organic solvent. The branched DNA complexes may comprise X-shaped DNA complexes, or Y-shaped DNA complexes, or T-shaped DNA complexes, or dendrimeric DNA complexes, and the like.

In this and other aspects of the invention relating to hydrogels and nanoparticles, the hydrogel or nanoparticle may further comprise a non-siRNA agent (i.e., an agent to be delivered to cells, tissues, or a subject, according to the invention, that is not siRNA in nature). Accordingly, the invention contemplates that hydrogels and nanoparticles may be used to deliver siRNA only or siRNA and one or more non-siRNA agents. The non-siRNA agent may be of any nature, including nucleic acid, provided it is not an siRNA as defined herein.

The non-siRNA agent may be a therapeutic agent, or an anti-cancer agent (such as but not limited to doxorubicin), or an immunostimulatory agent (such as but not limited to an immunostimulatory CpG nucleic acid), or a nucleic acid binding moiety (such as but not limited to doxorubicin, or a diagnostic agent, or an imaging agent, inter alia.

In some embodiments, the hydrogel further comprises a lipid coating. The lipid coating may comprise a heterogeneous mixture of lipids or a homogeneous mixture of lipids. The lipid coating may comprise anionic (negatively charged) lipids, or neutral lipids, or a combination of anionic and neutral lipids. Neutral lipids may be polar lipids or zwitterionic lipids. Therefore in some embodiments the lipids are preferably non-cationic lipids. In some embodiments, the lipid coating comprises neutral lipids and anionic lipids in a 4:1 molar ratio.

In some embodiments, the lipid coating comprises phospholipids including for example dioleoylphosphatidylcholine (DOPC) or dioleoylphosphatidylglycerol (DOPG), or a mixture of DOPC and DOPG. In some embodiments, the lipid coating comprises DOPC and DOPG in a 4:1 molar ratio.

In some embodiments, the hydrogel has dimensions ranging from about 1 micron to about 1000 microns. The hydrogel may be of any shape.

In some embodiments, the hydrogel comprises no organic solvent. In some embodiments, the hydrogel is dried. In other embodiments, the hydrogel is provided in a pharmaceutically acceptable carrier, optionally in a syringe or other device that facilitates in vivo administration.

In another aspect, the invention provides a method comprising crosslinking branched nucleic acids attached to R-form siRNA in the presence of a DNA ligase and optionally ATP, thereby forming a hydrogel.

As recited above, the branched nucleic acids may comprise X-shaped branched nucleic acids such as X-shaped DNA, or Y-shaped nucleic acids such as Y-shaped DNA, or T-shaped nucleic acid such as T-shaped DNA, or dendrimeric nucleic acids such as dendrimeric DNA, and the like. The branched nucleic acids may be homogeneous or heterogeneous as described above.

In some embodiments, individual branched nucleic acids are attached, whether covalently or non-covalently and whether directly or indirectly, to 1, 2, 3, 4, or more siRNA, preferably R-form siRNA. Such attachment typically occurs before crosslinking.

In some embodiments, the method further comprises coating the hydrogel with a lipid coating. The lipid coating may comprise a homogenous mixture of lipids, or it may comprise a heterogeneous mixture of lipids. In some embodiments, the lipid coating comprises non-cationic lipids. The lipid coating may comprise neutral (such as polar and zwitterionic) and/or anionic lipids. The neutral lipids may comprise DOPC, and the anionic lipids comprise DOPG.

In some embodiments, the method does not comprise the use of organic solvents, and thus the hydrogels formed lack organic solvents. In some embodiments, the method does not comprise cationic lipids or cationic polymers.

In another aspect, the invention provides a hydrogel produced by the method of any of the foregoing methods.

In another aspect, the invention provides a method comprising administering any of the foregoing complexes or any of the foregoing hydrogels or any of the hydrogels made according to any of the foregoing methods to a subject in need thereof in an effective amount.

In another aspect, the invention provides a method comprising reducing expression of a target protein in a cell by contacting the cell with any of the foregoing complexes or any of the foregoing hydrogels or any of the hydrogels made according to any of the foregoing methods, wherein the complex or hydrogel comprises a target-specific siRNA.

In another aspect, the invention provides a method comprising reducing expression of a target protein in vivo for a period of 2-3 days or 4-7 days following administration to a subject of any of the foregoing complexes or any of the foregoing hydrogels or any of the hydrogels made according to any of the foregoing methods, wherein the complex or hydrogel comprises a target-specific siRNA.

In another aspect, the invention provides a method comprising reducing expression of a target protein in vivo for a period of 2-3 days following administration to a subject of a DNA hydrogel that comprises target-specific siRNA

In some embodiments, the subject has or is at risk of developing cancer. In some embodiments, the subject has or is at risk of developing an infection. In some embodiments, the subject has or is at risk of developing an allergy or asthma. In some embodiments, the subject has or is at risk of developing a neurodegenerative disorder. In some embodiments, the subject has or is at risk of developing an autoimmune disorder.

In some embodiments, the hydrogel releases R-form siRNA for at least a day. In some embodiments, the hydrogel releases R-form siRNA over a period of about 3 days.

In some embodiments, the hydrogel is introduced in or near a tumor. In some embodiments, the hydrogel is introduced in an organ or tissue. The hydrogel may be administered locally or systemically.

In another aspect, the invention provides a method comprising combining in solution branched nucleic acid complexes, DNA ligase, ATP, and lipids to form a mixture comprising lipid-encapsulated and free unencapsulated branched nucleic acids complexes, wherein the branched nucleic acid complexes comprise branched nucleic acids linked to R-form siRNA, incubating the mixture under conditions and for a time sufficient for the DNA ligase to crosslink the branched nucleic acid complexes, removing the free unencapsulated branched nucleic acid complexes from the mixture before or after incubating the mixture, and harvesting remaining cross-linked branched nucleic acid complexes.

In some embodiments, the lipids are non-cationic phospholipids. In some embodiments, the harvested crosslinked branched nucleic acid complexes are lipid-encapsulated. In some embodiments, the method further comprises removing lipids from the mixture prior to harvesting remaining crosslinked branched nucleic acid complexes. In some embodiments, the harvested crosslinked branched nucleic acid complexes do not have a lipid coating. In some embodiments, the method further comprises size selecting the lipid-encapsulated branched nucleic acid complexes. In some embodiments, the method further comprises size selecting the remaining crosslinked branched nucleic acid complexes before or after harvest.

In some embodiments, the free branched nucleic acid complexes are removed from the mixture using a nuclease. The nuclease may be an exonuclease. The nuclease may be a DNase or an RNase.

In some embodiments, the lipids are removed using detergent or an enzyme. The detergent may be Triton-X. The enzyme may be a lipase such as a phospholipase.

In some embodiments, the branched nucleic acids comprise branched DNA. In some embodiments, they may comprise X-shaped nucleic acids such as X-shaped DNA, or Y-shaped nucleic acids such as Y-shaped DNA, or T-shaped nucleic acids such as T-shaped DNA, or dendrimeric nucleic acids such as dendrimer DNA, and the like. The branched nucleic acids may be a homogeneous mixture or they may be a heterogeneous mixture. In some embodiments, the branched nucleic acids comprise branched nucleic acids having at least 2 crosslinking ends.

In some embodiments, the DNA ligase is T4 DNA ligase. In some embodiments, the solution is an aqueous solution.

In some embodiments, the lipids comprise anionic lipids and/or neutral lipids. The neutral lipids and anionic lipids may be present in a 4:1 molar ratio. The lipids may comprise phospholipids. The lipids may be a homogeneous mixture or a heterogenous mixture of lipids. The lipids may comprise dioleoylphosphatidylcholine (DOPC) and/or dioleoylphosphatidylglycerol (DOPG). In some embodiments, DOPC and DOPG are present in a 4:1 molar ratio.

In some embodiments, the branched nucleic acid complexes comprise a non-siRNA agent, such as described above.

In another aspect, the invention provides a nanoparticle of crosslinked nucleic acids made according to any of the foregoing methods.

In another aspect, the invention provides a nanoparticle comprising crosslinked branched nucleic acid complexes comprising R-form siRNA and having a dimension (such as an average diameter or a longest diameter) in the range of about 100 nm to about 1 micron.

In some embodiments, the nanoparticle has a dimension (e.g., an average diameter or a longest diameter) ranging from about 100 nm to about 1 micron. In some embodiments, the nanoparticle has a dimension (e.g., an average diameter or a longest diameter) ranging from about 100 nm to about 500 nm.

In some embodiments, the nanoparticle is dried. In some embodiments, the nanoparticle is provided in a pharmaceutically acceptable carrier, and optionally in a delivery device such as a syringe.

In some embodiments, the nanoparticle comprises a lipid coating. In some embodiments, the nanoparticle lacks a lipid coating. In some embodiments, the nanoparticle comprises one or more internal lipid layers. The lipid coating or lipid layers may comprise anionic (negatively charged) lipids. The lipid coating or lipid layers may comprise homogeneous or heterogenous mixtures of lipids.

In some embodiments, the nanoparticle comprises branched DNA complexes comprising R-form siRNA. The branched DNA complexes may comprise non-covalently attached R-form siRNA or covalently attached R-form siRNA. Typically, the siRNA is not complexed with a cationic polymer or lipid.

In some embodiments, the crosslinked branched nucleic acid complexes comprise crosslinked X-shaped DNA, or crosslinked Y-shaped DNA, or crosslinked dendrimeric DNA. The branched nucleic acids may be heterogeneous or they may be homogeneous. The nanoparticle may not comprise organic solvent. In some embodiments, the nanoparticle comprises a non-siRNA agent, including but not limited to those described above.

In some embodiments, the nanoparticle is provided in a dry form, while in other embodiments it is provided in a pharmaceutically acceptable carrier.

In another aspect, the invention provides a method comprising administering any of the foregoing nanoparticles, or compositions comprising any of the foregoing nanoparticles, or nanoparticles produced by any of the foregoing methods to a subject in need thereof in an effective amount. Subjects include but are not limited to those recited above.

In some embodiments, the nanoparticles release siRNA over a period of about 3 days. In some embodiments, the nanoparticles release siRNA over a period of about 7 days. In some embodiments, the nanoparticles comprise a non-siRNA agent such as but not limited to those described above.

In some embodiments, the nanoparticles are administered systemically, including for example intravenously. In some embodiments, the nanoparticles are administered locally.

In another aspect, the invention provides a method comprising reducing expression of a target protein in vivo for a period of 2-3 days following administration to a subject of nanoparticles comprising crosslinked branched DNA complexes comprising target-specific siRNA.

In another aspect, the invention provides a method comprising reducing expression of a target protein in vivo for a period of about 7 days following administration to a subject of nanoparticles comprising crosslinked branched DNA complexes comprising target-specific siRNA.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF DRAWINGS

It is to be understood that the Figures are not necessarily to scale, emphasis instead being placed upon generally illustrating the various concepts discussed herein.

FIG. 1 illustrates a hydrogel network formed by crosslinking of X-DNA monomers functionalized with different cargos, such as reporter dyes, functional single- or double-stranded DNA oligonucleotides, DNA-binding drugs, etc.

FIG. 2 illustrates crosslinked nucleic acid nanoparticles. Left Panel: Schematic drawing. Upper Right: Confocal microscope image of nanoparticles labeled with SYBR dye. Lower Right Liquid cell AFM image of nanoparticle.

FIG. 3 diagrams an exemplary non-limiting synthesis process for the nanoparticles. Nanoparticles are constructed using a lipid “template”. A lipid film is mixed with branched nucleic acid monomers such as X-DNA monomers. Crosslinking agents such as T4 DNA enzyme are also included. The mixtures are put into a sonication probe (e.g., repeated with 5 to 1 watt in power) and immediately extruded under nanometer sized membrane filter (Step I). After one-day incubation, the mixture is first treated by an exonuclease and then centrifuged with 10% sucrose gradient in order to completely remove unencapsulated substrates such as free lipids and/or free nucleic acids (Step II). Optionally, the lipid coatings may be removed from the nanoparticles. To remove such lipid coatings, the nanoparticles are treated with either Triton X-100 and/or phospholipase. The resultant “naked” nanoparticles are collected using a high speed spin-down method (Step III).

FIG. 4 illustrates the design of X-DNA-siRNA hybrid structures functional for gene silencing. (Top) The siRNA molecules comprise a double-stranded RNA region flanked by two 3′ overhangs. In one embodiment, the R form siRNA comprises a 3′ overhang on its sense strand that is DNA in nature and a 3′ overhang on the antisense strand that is RNA in nature. When this form binds to an X-DNA (or other branched nucleic acid), the 3′ DNA overhang on the sense strand hybridizes to the overhang on the branched DNA. Conversely, in one embodiment, the L form siRNA comprises a 3′ overhang on its antisense strand that is DNA in nature and an RNA-based overhang on the 3′ end of the sense strand. When this form binds to an X-DNA (or other branched nucleic acid), the 3′ overhang on the antisense strand hybridizes to the overhang on the branched DNA. Other versions of L and R forms of siRNA are contemplated including those having one blunt end and one 3′ overhang end. (Bottom) 293T cells were co-transfected with firefly luciferase (Frluc), renilla luciferase (Rrluc) and different siRNA constructs. Luciferase signals were quantified 24 hrs later. Frluc/Rrluc value is normalized to that of cells treated with control siRNA that is not complementary to firefly luciferase (negative control). X and siR refer to X-DNA blocks and siRNA molecules, respectively.

FIG. 5 illustrates RNA interference of ‘X’ nanostructure DNA-RNA hybrids and DNA/siRNA-nanogels targeting GFP in melanoma cells. The study model cell line is B16-F0 expressing EGFP. DNA-RNA or RNA molecules are transfected with an in vitro lipid-based transfection reagent (Roche/Applied Science) into B16-F0 melanoma tumor cells.

FIG. 6 illustrates that siRNA/DNA-nanogels promote sustained gene silencing. DNA/siRNA-nanogels mixed with lipid transfection reagent (Roche/Applied Science) or free siRNA plus lipid transfection reagent were added to B16-F0 tumor cells expressing GFP, and GFP fluorescence was quantified over time. siRNA/DNA-nanogels elicit sustained gene silencing over several days while equivalent molar quantities of free siRNA achieved only very transient silencing, with GFP expression regained in B16-GFP cells fully restored by 48 hrs.

FIG. 7 illustrates that lipid-coated DNA/siRNA hybrid nanogels are avidly internalized by cells without toxicity. (Left) Internalization of lipid-coated DNA-nanogel nanoparticles (fluorescently labeled in blue) into B16-GFP melanoma cells, demonstrating robust uptake of the lipid-coated particles by tumor cells. (Right) Viability of B16-GFP cells 24 hrs after treatment with a range of lipid-coated DNA/siRNA nanogels.

FIG. 8 illustrates downregulation of GFP in B16F0-GFP tumor cells following treatment with lipid-coated DNA/siRNA nanogels carrying GFP-directed siRNA, compared to untreated control cells. (Top) Quantification of GFP fluorescence in cells at 24 hrs. (Bottom) Confocal micrographs of untreated melanoma cells (left two columns) and DNA/siRNA-nanogel treated cells (right two columns; nanogels fluorescence overlaid in blue). GFP expression is extinguished in the vast majority of treated cells.

### DETAILED DESCRIPTION OF INVENTION

The invention relates in its broadest sense to compositions and methods for delivering agents including siRNA to cells in vitro and in vivo, in some instances for extended periods of time.

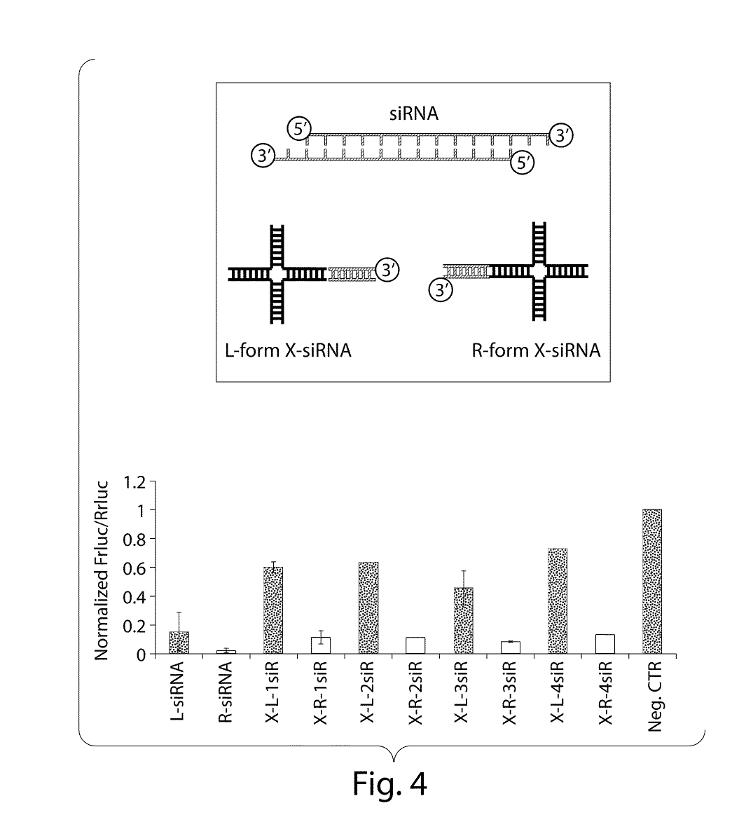

The invention is based in part on the surprising discovery that attaching siRNA to branched nucleic acids can impact siRNA activity, with one bound siRNA form retaining all or most of its activity after such attachment and another form having reduced activity after such attachment. More specifically, it has been found in accordance with the invention that when R-form siRNA is bound to a branched monomer it is active while when L-form siRNA is bound to an identical monomer its activity is partially and in some cases nearly completely reduced. FIG. 4 illustrates the difference between the two forms as bound to an X DNA monomer. An R form siRNA comprises a 3′ overhang on its antisense strand and it is this 3′ overhang that is “free” upon attachment to a branched nucleic acid. An L form siRNA comprises a 3′ overhang on its sense strand and it is this 3′ overhang that is “free” upon attachment to a branched nucleic acid. The other end of these siRNAs may be blunt ended or may have a 3′ DNA overhang to facilitate attachment to the branched nucleic acid. Although not intended to be bound by any particular theory or mechanism, bound R-form siRNA may be more accessible to the cellular machinery involved in siRNA processing and recognition, while bound L-form may not be. This may explain the disparity in the activity level of the two bound forms.

The invention contemplates generation of siRNA having defined ends (whether overhang or blunt, and whether RNA or DNA). Exemplary sequences are provided in Table 1. The invention further contemplates generation of branched nucleic acids. Again, exemplary sequences are provided in Table 1. Once formed, the siRNA and branched nucleic acids are combined in order to form a hybrid branched nucleic acid that comprises one or more siRNA arms. Attachment of the siRNA to the branched nucleic acid may occur through simple non-covalent hybridization or it may occur through enzyme-mediated ligation. Any siRNA can be generated in an R-form provided that it exhibits a free 3′ antisense end once bound to a branched nucleic acid.

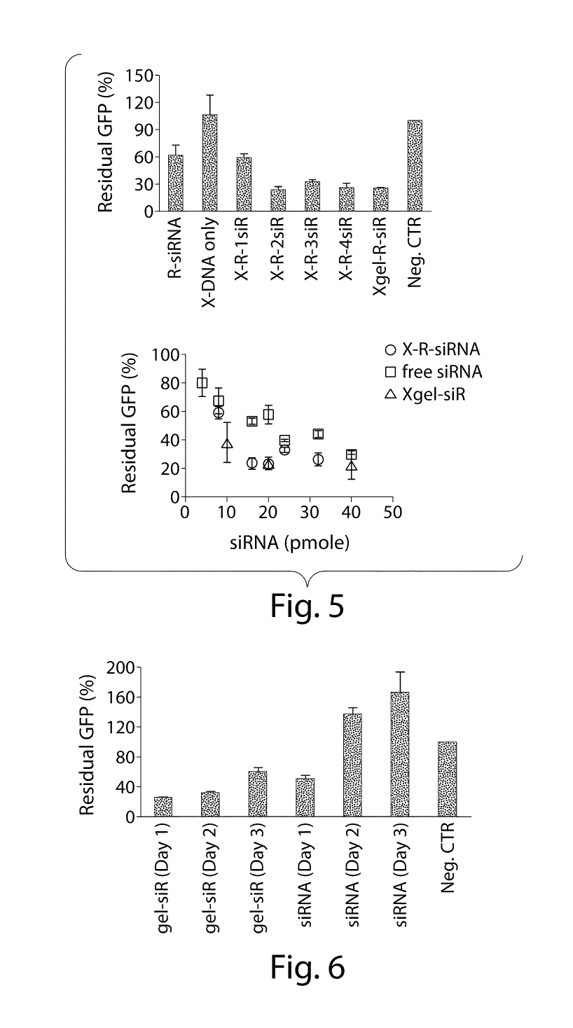

As stated above, branched monomers may by attached to 1, 2, 3, 4, or more RNA such as siRNA. The maximum number of siRNA per monomer will depend on the number of arms per monomer and whether the monomer is to be used in crosslinked or non-crosslinked form. As shown in FIG. 4, free X DNA monomers having 1, 2, 3, or 4 R-form siRNA were more effective than X DNA monomers having 1, 2, 3 or 4 L-form siRNA in down-regulating protein expression in transiently transfected cells in vitro. As shown in FIG. 5, free X DNA monomers having 2, 3 or 4 R-form siRNA were more effective than free R-form siRNA and as effective as a 250 nm nanoparticle made of crosslinked X DNA monomers each having one R-form siRNA at downregulating protein expression in a GFP expressing melanoma cell line.

The invention provides a variety of branched nucleic acid monomers complexed with R-form siRNA. As discussed in greater detail herein, branched nucleic acid monomers include X-shaped nucleic acids (e.g., X DNA), Y-shaped nucleic acids (e.g., Y-DNA), T-shaped nucleic acids (e.g., T DNA), dendrimer-shaped nucleic acids, dumbbell shaped nucleic acids, and the like. The invention further contemplates the use of such monomers to deliver siRNA to cells whether in vitro or in vivo for the purpose of modulating protein expression. Homogeneous or heterogeneous populations of these monomers may be used. The monomers may be used in an uncrosslinked form. In some instances, they may be used together with a lipid-based transfection reagent, such as X-tremeGENE™ which is commercially available from Roche/Applied Science (Catalog No. 04476093001).

The invention also provides crosslinked branched nucleic acids that comprise siRNA. These crosslinked forms may comprise siRNA in both R- and L-form for some applications, or only R-form siRNA for some other applications. In some instances, a mixture of R- and L-forms are used, with the majority of the siRNA being R-form. The crosslinked forms may be generated by incubating the branched nucleic acid monomers comprising siRNA in the presence of a crosslinking agent. The monomers may be attached, covalently or non-covalently, to a non-RNA agent or such agent may simply be present in the same aqueous solution as the monomers. The crosslinking agent is typically an enzyme such as a DNA ligase that acts on free “crosslinkable” ends of the branched monomers. The resultant hydrogels may be lipid coated or naked. They may be used with or without reagents that facilitate their uptake such as but not limited to transfection reagents (e.g., X-tremeGENE™ which is commercially available from Roche/Applied Science). As used herein, a hydrogel is a three dimensional matrix of crosslinked monomers that is able to retain water or other aqueous solution. These hydrogels may be produced (and/or extruded) in a variety of shapes and sizes depending on their intended use. In some important embodiments, the hydrogels take the form of nanoparticles.

The invention is also based in part on the discovery of a method for synthesizing submicron particles (referred to herein interchangeably as nanoparticles) comprising the aforementioned hydrogels and preferentially R-form siRNA. The particles generated by the methods of the invention are non-toxic, biodegradable and demonstrate a prolonged release profile, making them ideal carriers for siRNA in vivo. Nanoparticles may be taken up by cells and can continually release their active agent payload into such cells for an extended period of time.

It is to be understood that the monomers, hydrogels and/or nanoparticles may comprise other agents in addition to siRNA. It is further to be understood that the invention contemplates the intracellular and extracellular use of monomers, hydrogels and nanoparticles, both in vitro or in vivo.

siRNA

Small (or short) interfering RNAs (siRNA) are RNA molecules capable of causing interference and thus post-transcriptional silencing of specific genes in cells, including mammalian cells. siRNA comprise a double stranded region that is typically about 5-50 base pairs, more typically 10-40 base pairs, and even more typically 15-30 base pairs in length. The siRNA attached to the branched monomers may be 20-50, 25-50 or 30-40 base pairs in length. Again, without intending to be bound by any particular theory or mechanism, these siRNA may be digested by the RNase III Dicer to yield smaller siRNA in the range of 19-28 base pairs, including 19 base pairs, 21 base pairs, 23 base pairs, 25 base pairs, and 27 base pairs in length. It is known that siRNA in this size range can be incorporated into and acted upon by the enzyme complex called RNA-Induced Silencing Complex (RISC), with a net result of target RNA degradation and/or inhibition of any protein translation therefrom. In a similar manner, double-stranded RNAs with other regulatory functions such as microRNAs (miRNA) could also be incorporated into complexes comprising branched nucleic acids. Reference can be made to Bass, Nature 411: 428-29 (2001); Elbashir et al., Nature 411: 494-98 (2001); Fire et al., Nature 391: 806-11 (1998); WO 01/75164, and U.S. Pat. Nos. 6,506,559, 7,056,704, 7,078,196, 7,432,250, for greater detail on siRNA as well as methods of making siRNA.

siRNA forms such as the R- and L-form will have overhangs on one or both ends. As discussed herein, an R-form siRNA has a 3′ overhang on its antisense strand. It may be blunted on its other end and/or it may have a 3′ overhang on its other end, including an overhang comprising DNA residues. When bound to a branched nucleic acid, the 3′ antisense strand is the free end of the siRNA. Alternatively, an L-form siRNA has a 3′ overhang on its sense strand. It may be blunted on its other end and/or it may have a 3′ overhang on its other end, including an overhang comprising DNA residues. When bound to a branched nucleic acid, the 3′ sense overhang is the free end of the siRNA. The overhangs that hybridize to the branched nucleic acid, when they are used, may be 1, 2, 3, 4, 5, or more residues (e.g., DNA residues) in length. The overhangs that represent the free ends upon attachment to a branched nucleic acid may be 1, 2, 3, 4, 5, or more residues (e.g., RNA residues) in length.

Thus, the siRNA may be comprised of ribonucleotides or a combination of ribonucleotides and deoxyribonucleotides, including in some instances modified versions of one or both. For example, ribonucleotides containing a non-naturally occurring base (instead of a naturally occurring base) such as uridines and/or cytidines modified at the 5-position, e.g. 5-(2-amino)propyl uridine, 5-bromo uridine, or adenosines and/or guanosines modified at the 8-position, e.g. 8-bromo guanosine, or deaza nucleotides, e.g. 7-deaza-adenosine, or O- and N-alkylated nucleotides, e.g. N6-methyl adenosine can be incorporated into the siRNA. As another example, sugar-modified ribonucleotides having a 2′ OH-group replaced by a group selected from H, OR, R, halo, SH, SR, NH₂, NKR, NR₂ or CN, wherein R is C₁-C₆ alkyl, alkenyl or alkynyl and halo is F, Cl, Br or I. As yet another example, the backbone may be modified to comprise modified backbone linkages such as but not limited to phosphorothioates. The siRNA may comprise modifications at the base, sugar and/or backbone, including a variety of such modifications.

Thus, siRNA molecules can be provided as and/or derived from one or more forms including, e.g., as one or more isolated small-interfering RNA (siRNA) double stranded duplexes, as longer double-stranded RNA (dsRNA), or as siRNA or dsRNA transcribed from a transcriptional cassette in a DNA plasmid. The siRNA molecules may have overhangs (e.g., 3′ or 5′ overhangs as described in Elbashir et al., Genes Dev., 15:188 (2001) or Nykanen et al., Cell, 107:309 (2001)), or may lack overhangs (i.e., have blunt ends). The person of ordinary skill in the art will appreciate and understand how such starting sources may be modified in order to arrive at the R- and L-forms described herein.

Apart from the requirements set forth herein, the siRNA may be modified in one or more ways. As an example, the siRNA may be attached to a detectable label such as a fluorophore or an in vivo imaging label. Such labels may be used to assay release and/or retention of siRNA into a cell or an environment and may in some instances be used to determine the release characteristics of the hydrogels and nanoparticles of the invention.

siRNA are targeted to genes in vivo or in vitro if all or part of the nucleotide sequence of their duplex (or double stranded) is complementary to a nucleotide sequence of the targeted gene. siRNA made be synthesized based upon known (or predicted) nucleotide sequences of nucleic acids that encode proteins or other gene products. The sequence may be complementary to a translated or untranslated sequence in the target. Alternatively, siRNA may be synthesized using random sequences for example in order to screen siRNA libraries and/or to silence previously unknown genes. The degree of complementarity between the siRNA and the target may be 100% or less than 100%, provided that sufficient identity exists to a target to mediate target-specific silencing. The art is familiar with efficacious siRNA that are less than 100% complementary to their target.

It is to be understood that the invention is not limited with respect to the nature of the target gene. The art is familiar with a wide variety of siRNA for a wide variety of targets. The invention contemplates use of any such siRNA in the complexes described herein. Non-limiting and non-exhaustive examples of targets include nucleic acids that are upregulated in disorders including cancer, autoimmune, inflammatory or other abnormal immune-related disorders, neurodegenerative disorders, cardiac disorders, whether such upregulation is considered to cause or be a manifestation of the disorder, mutant nucleic acids the expression of which interferes with the activity of wild type proteins or the otherwise normal functioning of a cell (e.g., p53 or other oncogenes), and the like.

The level of silencing or interference may be measured in any number of ways, including quantitation of mRNA species and/or protein species. In some instances, mRNA quantitation is preferred particularly where the protein is intracellular or otherwise difficult to observe and/or assay. mRNA levels may be measured using RT-PCR or RACE, as an example. Protein levels may be measured using immunohistochemical staining. mRNA or protein levels may be reduced by 10%, 20%, 30%, 40%, 50%, 60%, 70%, 80%, 90%, 95%, 99%, or even 100%. Depending on the application, partial reduction (i.e., less than 100% may be sufficient) as compared to the level in the absence of the exogenously applied siRNA. In some embodiments, the level is reduced by 80% or more than 80% as compared to a control that has not been exposed to exogenously applied siRNA.

Some aspects of the invention provides prolonged (or extended or sustained) release of siRNA in vivo or in vitro. Therefore, in some instances, release and thus subsequent gene silencing occurs for days or weeks. For example, reduced expression of a target of interest may be observed for 1, 2, 3, 4, 5, 6, or 7 days, or for 1-2 weeks, or for longer periods of time.

### Branched Nucleic Acids

As used herein, branched nucleic acid are complexes comprising three or more nucleic acid strands in which some or all the strands hybridize to at least two other strands. Strands may comprise two regions (or sequences) each of which is complementary to regions (or sequences) of other strands. The complex may be “Y-shaped” if three strands contribute to the complex. Y-shaped nucleic acids (also referred to in the art and herein as Y nucleic acids) are described in greater detail in published US patent application US20050130180A1 to Luo et al. The complex may be “X-shaped” if four strands contribute to the complex. X-shaped nucleic acids (also referred to in the art and herein as X nucleic acids) are described in greater detail in published US patent application US20070148246A1 to Luo et al. In both instances, each strand in the complex hybridizes to two other strands. The branched nucleic acids may be dendrimeric nucleic acids, T-shaped nucleic acids, and/or dumbbell shaped nucleic acids, such as those illustrated and described in published US patent application US20050130180A1 to Luo et al. These references provide sequences of nucleic acids that may be used to produce branched nucleic acids such as but not limited to the Y- and X-shaped nucleic acids of the invention. In addition, these references provide sufficient guidance for how to select additional sequences to be used in the synthesis of such branched nucleic acids. Accordingly, these sequences and the rules governing the selection of these and other sequences are incorporated by reference herein in their entirety.

In some instances the generation of some of these nucleic acid forms may require one or more linear nucleic acids and some degree of ordered assembly of linear and branched nucleic acids. The art is however familiar with such processes and therefore they will not be described in any great detail herein. See for example published US patent applications US20050130180A1 and US20070148246A1, as well as Lee et al. Nat Biotech DOI:10.1038/NNANO.2009.93, 2009 (advance online publication); Um et al. Nat Materials 5(10): 797 (2006); Um et al. Nat Protocols 1(2):995-1000 (2006).

Luo et al. have reported the production of macroscopic three-dimensional hydrogels made from crosslinking of Y-shaped DNA and X-shaped DNA. See Um et al. Nat Materials 5(10): 797 (2006) and Park et al. Nat Materials 8: 432-437 (2009). The invention improves upon the report of Luo et al. in a number of ways. For example, the invention establishes that only R-form siRNA are active once bound to branched nucleic acids. This finding was not apparent or predictable from the teachings of Luo et al. As another example, the invention provides methods for generating nanoparticles which are more attractive and amenable for some applications than are the macroscopic gels of Luo et al. The submicron carriers of the instant invention therefore will find broader clinical use since they can be delivered to essentially any region of the body, and importantly can be taken up by cells where necessary.

The invention contemplates that individual branched nucleic acids may be comprised of DNA, RNA, PNA, LNA, combinations thereof, and modifications thereof. Individual complexes may be or they may not be “homogeneous” with respect to their nucleic acid make-up. That is, within an individual monomer, there may be base, sugar and backbone linkage variations. Homogeneous monomers may be used with other homogeneous (but different) complexes or with heterogeneous complexes. In some instances, monomers that are bound to RNA species such as siRNA may be referred to herein as DNA/RNA monomers.

The branched nucleic acids may be preformed or they may be formed from separate single-stranded nucleic acids. In the case of Y-shaped nucleic acids, typically three strands will be required each having complementarity to the other two strands. In the case of X-shaped nucleic acids, typically four strands will be required each having complementarity to at least two other strands.

The length of the single-stranded oligonucleotides will vary depending on the application. In some instances, the length of the oligonucleotide strands may be 5 or more nucleotides in length, and may range from 10-100 nucleotides (or 3.4-34 nanometers), while in others it may range from 100-1000 nucleotides (or 34-340 nanometers).

TABLE 1
Exemplary Sequences for X-DNA and siRNA

| | |
|---|---|
| RNA/DNA-(1)-x01 | 5′- CGA CCG ATG AAT AGC GGT CAG ATC CGT ACC TAC TCG TAG C -3′ (SEQ ID NO: 1) |
| RNA/DNA-(1)-x02 | 5′- /5Phos/CGA GTA GGT ACG GAT CTG CGT ATT GCG AAC GAC TCG -3′ (SEQ ID NO: 2) |
| RNA/DNA-(1)-x03 | 5′- CGA GTC GTT CGC AAT ACG GCT GTA CGT ATG GTC TCG -3′ (SEQ ID NO: 3) |
| RNA/DNA-(1)-x04 | 5′- CGA GAC CAT ACG TAC AGC ACC GCT ATT CAT CGG TCG -3′ (SEQ ID NO: 4) |
| RNA/DNA-(2)-x01 | 5′- CGA CCG ATG AAT AGC GGT CAG ATC CGT ACC TAC TCG TAG C -3′ (SEQ ID NO: 5) |
| RNA/DNA-(2)-x02 | 5′- /5Phos/CGA GTA GGT ACG GAT CTG CGT ATT GCG AAC GAC TCG TAG C -3′ (SEQ ID NO: 6) |
| RNA/DNA-(2)-x03 | 5′- /5Phos/CGA GTC GTT CGC AAT ACG GCT GTA CGT ATG GTC TCG -3′ (SEQ ID NO: 7) |
| RNA/DNA-(2)-x04 | 5′- CGA GAC CAT ACG TAC AGC ACC GCT ATT CAT CGG TCG -3′ (SEQ ID NO: 8) |
| RNA/DNA-(3)-x01 | 5′- CGA CCG ATG AAT AGC GGT CAG ATC CGT ACC TAC TCG TAG C -3′ (SEQ ID NO: 9) |
| RNA/DNA-(3)-x02 | 5′- /5Phos/CGA GTA GGT ACG GAT CTG CGT ATT GCG AAC GAC TCG TAG C -3′ (SEQ ID NO: 10) |
| RNA/DNA-(3)-x03 | 5′- /5Phos/CGA GTC GTT CGC AAT ACG GCT GTA CGT ATG GTC TCG TAG C -3′ (SEQ ID NO: 11) |
| RNA/DNA-(3)-x04 | 5′- /5Phos/CGA GAC CAT ACG TAC AGC ACC GCT ATT CAT CGG TCG -3′ (SEQ ID NO: 12) |
| RNA/DNA-(4)-x01 | 5′- /5Phos/CGA CCG ATG AAT AGC GGT CAG ATC CGT ACC TAC TCG TAG C -3′ (SEQ ID NO: 13) |
| RNA/DNA-(4)-x02 | 5′- /5Phos/CGA GTA GGT ACG GAT CTG CGT ATT GCG AAC GAC TCG TAG C -3′ (SEQ ID NO: 14) |
| RNA/DNA-(4)-x03 | 5′- /5Phos/CGA GTC GTT CGC AAT ACG GCT GTA CGT ATG GTC TCG TAG C -3′ (SEQ ID NO: 15) |
| RNA/DNA-(4)-x04 | 5′- /5Phos/CGA GAC CAT ACG TAC AGC ACC GCT ATT CAT CGG TCG TAG C -3′ (SEQ ID NO: 16) |
| pGL3-L-siRNA_ss | 5′- /5Phos/rGrUrG rCrGrC rUrGrC rUrGrG rUrGrC rCrArA rCrUrU -3′ (SEQ ID NO: 17) |
| pGL3-L-siRNA_as | 5′- rGrUrU rGrGrC rArCrC rArGrC rArGrC rGrCrA rCGC TA -3′ (SEQ ID NO: 18) |
| pGL3-R-siRNA_ss | 5′- /5Phos/rGrUrG rCrGrC rUrGrC rUrGrG rUrGrC rCrArA rCGC TA -3′ (SEQ ID NO: 19) |
| pGL3-R-siRNA_as | 5′- /5Phos/rGrUrU rGrGrC rArCrC rArGrC rArGrC rGrCrA rCrUrU -3′ (SEQ ID NO: 20) |
| B16-GFP-siRNA_ss | 5′- /5Phos/rGrCrA rArGrC rUrGrA rCrCrC rUrGrA rArGrU rUGC TA -3′ (SEQ ID NO: 21) |
| B16-GFP-siRNA_as | 5′- /5Phos/rArArC rUrUrC rArGrG rGrUrC rArGrC rUrUrG rCrUrU -3′ (SEQ ID NO: 22) |

/5Phos/ means “5′ phosphorylation” on the oligomer.

### Hydrogels

Branched nucleic acid monomers are crosslinked to form hydrogels. Crosslinking typically occurs at the ends of the branched monomers (i.e., the arm ends) rather than randomly throughout the length of the nucleic acids. In this way, the pore size of the resultant crosslinked matrix can be controlled and also tailored for agents of various sizes and molecular weights.

Pore size of the hydrogel is dependent in part on the length of the arms in the branched nucleic acids. Generally longer starting oligonucleotide strands result in longer arms, which in turn result in larger pore sizes. This is because the branched nucleic acids crosslink with each other at their ends rather than randomly throughout their length. This ordered crosslinking allows the user to control the pore size of the resulting gels and thus to design nanoparticles suitable for particular payloads whether such payloads are small molecules or high molecular weight proteins.

As an illustration, assume an X-shaped nucleic acid having 4 arms of roughly equal length, made of strands that are each about 100 nucleotides in length. Taking in account that some nucleotides exist at the center of the X-shaped monomer and therefore do not contribute significantly to the length of the arm, each arm may have a length of about 45 nucleotides, and crosslinking two such arms together will yield dimensions of about 90 nucleotides in length. A pore may then have dimensions of 90 nucleotides by 90 nucleotides by 90 nucleotides (or about 31 nm by 31 nm by 31 nm, or about 30,000 nm³).

Pore size of the hydrogel is also dependent in part on the degree of crosslinking between monomers or the number of crosslinkable ends available in the population of monomers. As stated earlier, crosslinking occurs at the end of the arms of branched nucleic acids, although not typically at siRNA. In the absence of conjugated agents, X-shaped nucleic acids have 4 arms available for crosslinking, Y-shaped nucleic acids have 3 arms available for crosslinking, and dendrimeric nucleic acids have multiple arms available for crosslinking. In the presence of agents, some of those arms may be occupied and thus not available for crosslinking. At least some of the monomers contributing to a crosslinked gel will have 3 or more crosslinkable arms in order to form a gel or network rather than an extended linear nucleic acid polymer. It will be understood that, if plurality of different monomers is used to generate the crosslinked matrix, these may differ in the number of crosslinkable arms, provided that at least some have three or more available arms. As an example, a mixture of X-shaped DNA monomers may be used and the mixture may comprise proportions of branched nucleic acids that comprise 1, 2, 3 or 4 crosslinkable sites, with the remaining sites available for conjugation to agent.

In some instances, the monomers used to generate the crosslinked gel have a uniform number of arms available for crosslinking. This approach is expected to yield a more uniform and predictable pore size. That is, in some cases all the monomers have three arms, and in other instances all the monomers have four arms, etc.

In some instances, pore size (diameter) may be in the range of 1-5 nm, 1-10 nm, 1-50 nm, or 1-100 nm, including about 1 nm, about 5 nm, about 10 nm, about 20 nm, about 30 nm, about 40 nm or about 50 nm.

### Nanoparticles

In some aspects of the invention, the hydrogels are produced as nanoparticles using the methods provided herein. Briefly, nanoparticles are produced by first encapsulating branched nucleic acid monomers in a liposome-like shell together with a crosslinking agent such a DNA ligase, followed by crosslinking the encapsulated monomers, and then optionally removing the liposome-like shell. RNA-based agents (such as siRNA) and non-RNA-based agents may also be encapsulated in the liposome-like shell simply by including such agents in the lipid/monomer aqueous solution prior to encapsulation. The resultant nanoparticles (or nanogels as the terms are referred to herein interchangeably) may have a lipid coating or they may be uncoated (or naked).

The nanoparticles of the invention possess one or more improved characteristics as compared to existing liposome and nanoparticle technology. First, the particles may be synthesized in aqueous conditions without the use of organic solvents. This means that small molecule drugs or proteins may be retained in a native state with higher activity levels than may otherwise be possible using most existing strategies. Second, the crosslinked hydrogel core of the nanoparticles can be manipulated to achieve a predictable and defined porosity based primarily on the length of the arms of the branched nucleic acids and the number of crosslinkable arms per branched nucleic acid monomer. The ability to control the porosity of the nucleic acid network allows the release rate of entrapped agents to be controlled in turn. Third, the nanoparticles may comprise free uncrosslinked arms that are coupled (or attached) to agents being delivered including siRNA as well as therapeutic agents, imaging agents, or sensing agents. Fourth, in some instances the nucleic acids used to generate the crosslinked gel may themselves be the agent being delivered rather than simply the scaffolding that carries and retains an agent. As an example, the nucleic acids may comprise immunostimulatory oligonucleotides (e.g., CpG oligonucleotides). Nanoparticles generated according to the methods of the invention exhibit loading and prolonged release of the chemotherapy drug doxorubicin and ovalbumin protein (data not shown).

The invention therefore provides inter alia methods of making nucleic acid based nanoparticles, the nanoparticles themselves as well as compositions comprising such nanoparticles, and methods of using such nanoparticles.

As used herein, nanoparticle refers to any particle having an average diameter in the range of 1 to 1000 nanometers (i.e., 1 micron). In some instances, such particles will have an average diameter in the range of 50 to 1000 nanometers, 50 to 900 nanometers, 50 to 800 nanometers, 50 to 700 nanometers, 50 to 600 nanometers, 50 to 500 nanometers, 50 to 400 nanometers, 50 to 300 nanometers, 50 to 200 nanometers, and/or 50 to 100 nanometers. The lower end of these ranges may alternatively be about 100 nanometers.

The nanoparticle may be of any shape and is not limited to a perfectly spherical shape. As an example, it may be oval or oblong. As a result, its size is referred to in terms of average diameter. As used herein, average diameter refers to the average of two or more diameter measurements. The dimensions of the microparticle may also be expressed in terms of its longest diameter or cross-section.

The nanoparticle comprises a crosslinked nucleic acid core. The crosslinked nucleic acids therefore create a three-dimensional mesh, network or gel. Accordingly, the nanoparticles are referred to herein interchangeably as nanogels. This crosslinked nucleic acid core may act as a scaffold for retaining agent(s) and/or it may comprise agent(s) itself.

It is to be understood that the invention contemplates the use of lipid-coated as well as uncoated nanoparticles, as illustrated in the Examples. The composition of the lipid coating will depend upon the lipids used to generate the nanoparticles in the first instance. Thus, the lipid coating, if present, may comprise neutral lipids and/or anionic lipids and/or other lipid membrane components (e.g., cholesterol, sphingomyelin, etc.) in varying molar ratios, and such lipids may be further conjugated to other moieties such as but not limited to PEG.

The nanoparticle release profile may vary depending on the nature and amount of agent, the nature of the nanoparticles themselves including whether or not they comprise a lipid coating, the size of the nanoparticles, the environment to which the nanoparticles are exposed, and the like. However, notwithstanding these various parameters, the nanoparticles are able to release siRNA for at least 3 days, at least 4 days, at least 5 days, at least 6 days, at least 7 days, at least 8 days, at least 9 days, at least 10 days, at least 11 days, at least 12 days, at least 13 days, at least 14 days, or longer. This sustained release profile allows for gene silencing for periods of time that exceed those possible with fee siRNA. In some instances, the nanoparticles are able to release other agents at appreciable and medically significant levels for at least 7 days, at least 14 days, at least 21 days, at least 28 days, at least 35 days, or longer. In some instances, the nanoparticles are able to release agent at appreciable levels for 1-3 days. This latter release profile may be suitable for vaccination purposes. The release profile may also be defined by the rate at which the agent is being released (agent weight/time) and/or the total amount of agent released.

### Methods of Making Nanoparticles

Generally, the nanoparticles are produced by mixing lipids with branched nucleic acids attached to siRNA, in the presence of agents that crosslink the nucleic acids. The lipids form liposome-like particles that encapsulate the branched nucleic acids. Crosslinking agents are also encapsulated in the lipid particles and thus are able to act upon the nucleic acids. The nanoparticles may also contain agents intended for use in vivo or in vitro including without limitation therapeutic agents and diagnostic agents. These agents are typically included in the mixture of lipids and branched nucleic acids and in some instances may be combined with the branched nucleic acids prior to contact with the lipids. The relatively mild conditions used to generate nanoparticles ensure that the activity of the delivered agent will not be compromised significantly (if at all) during the process.

In one embodiment, the lipids are rehydrated in an aqueous solution with the branched nucleic acids. The method does not require the use of organic solvents and therefore the resultant nanoparticles are free of organic solvents (such as chloroform, dichloromethane, acetone and the like) that would render the nanoparticles toxic and unsuitable for in vivo use.

As discussed herein, the nanoparticles may be synthesized with a single type of branched nucleic acid or a combination of branched nucleic acids. Similarly, a single type of lipid may be used or a combination of lipids may be used. The types of branched nucleic acids, the number of sites available for crosslinking, the number of sites available for carrying payload, and the types and ratios of lipids may all be varied in accordance with the invention.

The lipids, branched nucleic acids, crosslinking agents and typically agents intended for delivery are mixed (e.g., sonicated) in order to disperse the lipids and produce liposome-like particles. Sonication times may vary but it is expected that repeated pulses lasting in duration of a few seconds, to a few minutes (depending on the volume and lipid density) will suffice. The mixture is expected to contain liposome-like particles comprising internal branched nucleic acids and crosslinking agent, empty liposome-like particles, free unencapsulated nucleic acids, and free crosslinking agent. As discussed in greater detail herein, the ratio of lipid to nucleic acid can impact the size of nanoparticles generated, with larger lipid to nucleic acid ratio tending to produce smaller particles. Ratios in the range of 200:1 to 5:1, or in the range of 100:1 to 5:1, or in the range of 100:1 to 10:1, or in the range of 50:1 to 10:1 can be used.

Following this step therefore the branched nucleic acids will either be encapsulated or free. As used herein, free branched nucleic acids refer to unencapsulated nucleic acids. These may exist as individual monomers or as crosslinked nucleic acids.

One step in the synthesis process requires that the entire mixture or an enriched fraction that contains the nucleic acid bearing liposome-like particles be subjected to conditions sufficient for crosslinking to occur. Such conditions and times will depend upon the type of crosslinking agent used. If the crosslinking agent is an enzyme, then the mixture can be incubated typically at neutral pH. It is expected that incubation on the order of several hours at a temperature in the range of 4-37° C. will suffice. The Examples demonstrate incubation for 24 hours at 16° C.

The synthesis process optionally includes steps to select nanoparticles of a certain size (and more likely size range). Size selection may be achieved using one or more filtration steps including for example passage through filtration membranes of decreasing pore size. Particles may be harvested from the membrane itself or from the run-through, depending on the desired size. Size selection may also be achieved using buoyant density gradient centrifugation, as well as other methods, as the invention is not limited in this regard. The particles may be selected having an average diameter in the range of 1-100 nm, 100-500 nm, 500-1000 nm, 1-1000 nm, or 100-1000 nm.

The synthesis process also typically includes steps to remove unreacted substrates and unwanted byproducts of the reaction. Unencapsulated nucleic acids may be removed by any means including chemical means (e.g., acid hydrolysis), enzymatic means (e.g., nuclease digestion such as but not limited to exonuclease digestion), and/or mechanical means (e.g., centrifugation). This may occur before or after the crosslinking step, and/or before or after size selection. Empty liposome-particles may be removed by any means including chemical means (e.g., detergent treatment such as Triton-X-100 treatment), enzymatic means (e.g., lipases such as phospholipases), and/or mechanical mans (e.g., centrifugation). These empty particles may be degraded at the same time as the lipid coating on the nucleic acid nanogels is removed, or it may occur separately. Typically lipid removal occurs following crosslinking in order to maintain the integrity of the nanogels.

The nanoparticles may be harvested at one or more steps in the synthesis process. As used herein, harvested means that the nanoparticles are collected and in some instances enriched by removal of other constituents of their environment (e.g., empty liposome-like particles or free branched nucleic acids).

The nanoparticles may be further modified or manipulated post-synthesis for example by addition of a label (e.g., for tracking or visualization). The label may be a fluorophore, or any other label that may be detected in vivo or in vitro as the particular application may require.

The method is not intended to be limited in these regards as the steps may be carried out in any manner that is convenient and suitable.

Lipids

Lipids are used in the invention in order to coat hydrogels, where desired. They are also used to form nanoparticles. In order to form nanoparticles, nucleic acids are encapsulated within lipid particles. The lipids may be isolated from a naturally occurring source or they may be synthesized apart from any naturally occurring source. The lipids may be amphipathic lipids having a hydrophilic and a hydrophobic portion. The hydrophobic portion typically orients into a hydrophobic phase, while the hydrophilic portion typically orients toward the aqueous phase. The hydrophilic portion may comprise polar or charged groups such as carbohydrates, phosphate, carboxylic, sulfato, amino, sulfhydryl, nitro, hydroxy and other like groups. The hydrophobic portion may comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

Typically, the lipids are phospholipids, though other lipid membrane components such as cholesterol, sphingomyelin, cardiolipin, etc. may also be additionally or alternatively used. Phospholipids or other lipids having the ability to form spherical bilayers capable of encapsulating nucleic acids can be used in the methods provided herein. Phospholipids include without limitation phosphatidylcholine, phosphatidylethanolamine, phosphatidylglycerol, phosphatidylinositol, phosphatidylserine, and the like.

The lipids may be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphosphatidylcholine (DMPC), and dioleoylphosphatidylserine (DOSE). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids in order to exclude cationic lipids from the class. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE),1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. Noncationic lipids also include polyethylene glycol-based polymers such as PEG 2000, PEG 5000 and polyethylene glycol conjugated to phospholipids or to ceramides (referred to as PEG-Cer).

In some instances, modified forms of lipids may be used including forms modified with detectable labels such as fluorophores and/or reactive groups such as maleimide (e.g., dioleoyl-phosphatidylethanolamine 4-(N-maleimidomethyl)-cyclohexane-1-carboxylate (DOPE-mal) and 1,2-dipalmitoyl-sn-glycero-3-phosphoethanolamine-N-[4-(p-maleimidophenyl)butyramide] (MBP)), among others. In some instances, the lipid is a lipid analog that emits signal (e.g., a fluorescent signal). Examples include without limitation 1,1′-dioctadecyl-3,3,3′,3′-tetramethylindotricarbocyanine iodide (DiR) and 1,1′-dioctadecyl-3,3,3′,3′-tetramethylindodicarbocyanine (DiD).

The invention contemplates the use of single lipids (referred to herein as homogeneous lipids) or combinations of lipids (referred to herein as heterogeneous lipids). If combinations are used, they may be combinations of anionic lipids, combinations of neutral lipids, or combinations of anionic and neutral lipids. Such combinations may be made from a range of molar ratios. For example, neutral lipids and anionic lipids may be used in molar ratios that range from 1:100 to 100:1, or in a range from 1:10 to 10:1 or in range from 1:1 to 10:1.

In one important embodiment, the lipids are combinations of zwitterionic lipids (such as DOPC) and anionic lipids (such as DOPG). As shown in the Examples, a 4:1 molar ratio of DOPC:DOPG resulted in more efficient internalization of a nanogels by melanoma cells in vitro in the absence of toxicity.

The lipids are preferably not conjugated to polyethylene glycol (PEG) prior to nanoparticle synthesis. As shown in the Examples, PEG-conjugated phospholipids appear to reduce the yield of nanoparticles in the methods described herein. However, since PEGylation is used clinically to increase the half-life of various agents including STEALTH liposomes, the instant invention contemplates modification of nanoparticles post-synthesis with PEG. This can be accomplished by using phospholipids with reactive groups (or functionalities) on their head groups (i.e., on the phosphate end) and then reacting such groups with PEG (or suitably modified PEG) post-synthesis. Reactive groups include without limitation amino groups such as primary and secondary amines, carboxyl groups, sulfhydryl groups, hydroxyl groups, aldehyde groups, azide groups, carbonyls, maleimide groups, haloacetyl (e.g., iodoacetyl) groups, imidoester groups, N-hydroxysuccinimide esters, and pyridyl disulfide groups.

The invention further contemplates using polymersome-forming block co-polymers having hydrophilic and hydrophobic blocks. Such block co-polymers can form liposome-like vesicles that entrap the branched nucleic acids and other components.

Crosslinking Agents

Crosslinking agents useful in the invention typically are able to conjugate nucleic acids to each other. In some instance, such conjugation is more specific and involves the ligation of double-stranded breaks. They include enzymes such as ligases that covalently bind nucleic acid ends to each other. In an even more specific example, crosslinking creates a phosphodiester bond between a 3′ hydroxyl of one nucleotide (and on one arm of a branched nucleic acid monomer) and a 5′ phosphate of another nucleotide (on the arm of another branched nucleic acid monomer). Exemplary enzymes include T4 DNA ligase, Thermus thermophilus ligase, Thermus acquaticus ligase, E. coli ligase, and Pyrococcus ligase. These and other enzymes may be used alone or in combination. Ligation carried out by enzymes is typically carried out between 4-37° C. Since the nanoparticles are intended for in vivo use in some instances, it is important that the crosslinking agents (and any other entities) present in or on the nanoparticles be non-toxic.

The invention further contemplates the use of nucleic acids including branched nucleic acids that are functionalized at their ends in order to effect crosslinking. For example, the nucleic acids may be used that comprise complementary chemical reactive groups (such as acrylate and amine) that would crosslink to each other through for example Michael addition, disulfide formation between thiolated nucleic acids, or other water-compatible crosslinking reactions, of which a variety are known in the art.

Nucleic Acids

The nucleic acid used to generate the branched monomers, hydrogels and nanoparticles may comprise naturally occurring and/or non-naturally occurring nucleic acids. If naturally occurring, the nucleic acids may be isolated from natural sources or they may be synthesized apart from their naturally occurring sources. Non-naturally occurring nucleic acids are synthetic.

The terms “nucleic acid” and “oligonucleotide” are used interchangeably to mean a polymer comprising multiple nucleotides (i.e., molecules comprising a sugar (e.g. a deoxyribose) linked to a phosphate group and to an exchangeable organic base, which is either a pyrimidine (e.g., cytosine (C), thymidine (T) or uracil (U)) or a purine (e.g., adenine (A) or guanine (G)).

Nucleic Acid Modifications

The monomers, hydrogels and nanoparticles may comprise DNA, modified DNA, and combinations thereof. Modifications may occur at the base, sugar and/or backbone. The backbone of oligonucleotides used to generate the branched nucleic acids may be homogeneous or heterogeneous (i.e., chimeric) backbone. The backbone may be a naturally occurring backbone such as a phosphodiester backbone or it may comprise backbone modification(s). In some instances, backbone modification results in a longer half-life for the oligonucleotides due to reduced nuclease-mediated degradation. This is turn results in a longer half-life and extended release profiles of the crosslinked complexes. Examples of suitable backbone modifications include but are not limited to phosphorothioate modifications, phosphorodithioate modifications, p-ethoxy modifications, methylphosphonate modifications, methylphosphorothioate modifications, alkyl- and aryl-phosphates (in which the charged phosphonate oxygen is replaced by an alkyl or aryl group), alkylphosphotriesters (in which the charged oxygen moiety is alkylated), peptide nucleic acid (PNA) backbone modifications, locked nucleic acid (LNA) backbone modifications, and the like. These modifications may be used in combination with each other and/or in combination with phosphodiester backbone linkages.

Alternatively or additionally, the oligonucleotides may comprise other modifications including modifications at the base or the sugar moieties. Examples include nucleic acids having sugars which are covalently attached to low molecular weight organic groups other than a hydroxyl group at the 3′ position and other than a phosphate group at the 5′ position (e.g., a 2′-O-alkylated ribose), nucleic acids having sugars such as arabinose instead of ribose. Nucleic acids also embrace substituted purines and pyrimidines such as C-5 propyne modified bases (Wagner et al., Nature Biotechnology 14:840-844, 1996). Other purines and pyrimidines include but are not limited to 5-methylcytosine, 2-aminopurine, 2-amino-6-chloropurine, 2,6-diaminopurine, hypoxanthine. Other such modifications are well known to those of skill in the art.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863, and alkylphosphotriesters (in which the charged oxygen moiety is alkylated as described in U.S. Pat. No. 5,023,243 and European Patent No. 092574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Nucleic acids can be synthesized de novo using any of a number of procedures known in the art including for example the b-cyanoethyl phosphoramidite method (Beaucage and Caruthers Tet. Let. 22:1859, 1981), and the nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated nucleic acid synthesizers available in the market. These nucleic acids are referred to as synthetic nucleic acids.

Alternatively, oligonucleotides may be generated from larger nucleic acids such as but not limited to plasmids. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acid. An isolated nucleic acid generally refers to a nucleic acid which is separated from components which it is normally associated with in nature. As an example, an isolated nucleic acid may be one which is separated from a cell, from a nucleus, from mitochondria, or from chromatin.

Agents

In addition to siRNA, the monomers, hydrogels and nanoparticles may contain agents that are intended for use in vivo and/or in vitro. As used herein, an agent is any atom, molecule or compound that can be used to provide benefit to a subject (including without limitation prophylactic or therapeutic benefit) or that can be used for diagnosis and/or detection (for example, imaging) in vivo, or that may be used for effect in an in vitro setting (for example, a tissue or organ culture, a clean up process, and the like). The agents may be without limitation therapeutic agents and diagnostic agents. Non-exhaustive lists are provided below.

The agents may be covalently or non-covalently attached to the crosslinked nucleic acids. If covalently or non-covalently attached, in some instances, the agents may be combined with the branched nucleic acids prior to contact with the lipids. Covalent attachment of agents to branched nucleic acids may involve the use of bonds that can be cleaved under physiological conditions or that can be caused to cleave specifically upon application of a stimulus such as light, whereby the agent can be released. Readily cleavable bonds include readily hydrolyzable bonds, for example, ester bonds, amide bonds and Schiff's base-type bonds. Bonds which are cleavable by light are known. In certain instances, the agent may be inactive in its bound form and activated only when released.

Non-covalently attached agents include those having affinity for nucleic acids (and thus having nucleic acid binding activity). Examples of such agents include without limitation certain drugs including certain cancer chemotherapies that act by binding to and damaging DNA, certain proteins (such as DNA repair enzymes, DNA polymerases, restriction endonucleases, topoisomerases, telomerases, and the like), nucleic acids or nucleic acid derivatives (e.g., PNA) that bind to other nucleic acids via Watson-Crick binding and/or Hoogsteen binding, non-nucleic acid probes that bind in the major and/or minor groove of the nucleic acid, and the like. The Examples illustrate the encapsulation of doxorubicin, an anti-cancer agent that binds DNA. Alternatively, the agents may be physically entrapped in the crosslinked nucleic acids, typically as a result of their size relative to the “pore” or “mesh” size of the resulting crosslinked nucleic acids.

Nanoparticles made in accordance with the methods described herein possess long-term release profiles for small molecule agents such as doxorubicin as well as higher molecular weight proteins such as ovalbumin (data not shown) The mechanism by which agents are released from the nanoparticle will depend in part on the mechanism by which the agent is retained in the nanoparticle in the first instance.

In one instance, the agent may be entrapped within the gel in the absence of covalent or non-covalent bonds. In this situation, degradation of the gel (and nucleic acids) in whole or in part must occur in order to release the agent. Degradation of the gel resulting in greater pore size can be another route through which the agents are released. This may be the case for example with high molecular weight agents such as proteins.

In another instance, the agent may be non-covalently attached to the crosslinked nucleic acids, and release from the nanoparticles may occur as the agent dissociates from the nucleic acids or functional or reactive groups on the nucleic acids. Since the nanoparticles are likely to be hydrated, the agent may simply diffuse away from its reactive site, into the aqueous solution, and out of the nanoparticle. If the agent is retained in the nanoparticle by virtue of its ability to bind to nucleic acids (e.g., it is a nucleic acid binding agent), a similar process is envisioned whereby the agent will dissociate from the nucleic acid and then diffuse out of the nanoparticle whether or not the nucleic acid gel has degraded. In an alternative manner, the nucleic acid gel may degrade, leaving the nucleic acid binding agent without a binding partner and able to diffuse out of the nanoparticle.

If the agent is covalently bound to the nucleic acid gel, then its release may come about by degradation of the gel. Alternatively, if the covalent bond is cleavable in response to physiological stimuli, then the agent may be released through cleavage of such bond. In either situation, it is possible that the agent may retain a part of the nucleic acid gel or the bond constituents but it is not expected that either will negatively impact the activity of the agent or be toxic to the subject.

The invention contemplates in some aspects the delivery of agents either systemically or to localized regions, tissues or cells. Any agent may be delivered using the methods of the invention provided that it can be loaded into the nanoparticles provided herein and can withstand the synthesis processes described herein. Since such processes are relatively innocuous, it is expected that virtually any agent may be used provided it can be encapsulated in the nanoparticles provided herein.

The nanoparticles may be synthesized and stored in, for example, a lyophilized and optionally frozen form. The agents should be stable during such storage procedures and times.

The agents may be naturally occurring or non-naturally occurring. Naturally occurring agents include those capable of being synthesized by the subjects to whom the nanoparticles are administered. Non-naturally occurring are those that do not exist in nature normally, whether produced by plant, animal, microbe or other living organism.

The agent may be without limitation a chemical compound including a small molecule, a protein, a polypeptide, a peptide, a nucleic acid, a virus-like particle, a steroid, a proteoglycan, a lipid, a carbohydrate, and analogs, derivatives, mixtures, fusions, combinations or conjugates thereof. The agent may be a prodrug that is metabolized and thus converted in vivo to its active (and/or stable) form. The invention further contemplates the loading of more than one type of agent in a nanoparticle and/or the combined use of nanoparticles comprising different agents.

One class of agents is peptide-based agents such as (single or multi-chain) proteins and peptides. Examples include antibodies, single chain antibodies, antibody fragments, enzymes, co-factors, receptors, ligands, transcription factors and other regulatory factors, some antigens (as discussed below), cytokines, chemokines, hormones, and the like.

Another class of agents that can be delivered using the nanoparticles of the invention includes chemical compounds that are non-naturally occurring.

A variety of agents that are currently used for therapeutic or diagnostic purposes can be delivered according to the invention and these include without limitation imaging agents, immunomodulatory agents such as immunostimulatory agents and immunoinhibitory agents (e.g., cyclosporine), antigens, adjuvants, cytokines, chemokines, anti-cancer agents, anti-infective agents, nucleic acids, antibodies or fragments thereof, fusion proteins such as cytokine-antibody fusion proteins, Fc-fusion proteins, analgesics, opioids, enzyme inhibitors, neurotoxins, hypnotics, anti-histamines, lubricants, tranquilizers, anti-convulsants, muscle relaxants, anti-Parkinson agents, anti-spasmodics, muscle contractants including channel blockers, miotics and anti-cholinergics, anti-glaucoma compounds, modulators of cell-extracellular matrix interactions including cell growth inhibitors and anti-adhesion molecules, vasodilating agents, inhibitors of DNA, RNA or protein synthesis, anti-hypertensives, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, targeting agents, neurotransmitters, proteins, cell response modifiers, and vaccines.

Imaging Agents. As used herein, an imaging agent is an agent that emits signal directly or indirectly thereby allowing its detection in vivo. Imaging agents such as contrast agents and radioactive agents that can be detected using medical imaging techniques such as nuclear medicine scans and magnetic resonance imaging (MRI). Imaging agents for magnetic resonance imaging (MRI) include Gd(DOTA), iron oxide or gold nanoparticles; imaging agents for nuclear medicine include ²⁰¹T1, gamma-emitting radionuclide 99 mTc; imaging agents for positron-emission tomography (PET) include positron-emitting isotopes, (18)F-fluorodeoxyglucose ((18)FDG), (18)F-fluoride, copper-64, gadoamide, and radioisotopes of Pb(II) such as 203 Pb, and 11In; imaging agents for in vivo fluorescence imaging such as fluorescent dyes or dye-conjugated nanoparticles. In other embodiments, the agent to be delivered is conjugated, or fused to, or mixed or combined with an imaging agent.

Immunostimulatory Agents. As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod, imidazoquinoline, resiquimod, nucleic acids comprising an unmethylated CpG dinucleotide, monophosphoryl lipid A or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

Antigens. The antigen may be without limitation a cancer antigen, a self antigen, a microbial antigen, an allergen, or an environmental antigen. The antigen may be peptide, lipid, or carbohydrate in nature, but it is not so limited.

Cancer Antigens. A cancer antigen is an antigen that is expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)—0017-1A/GA733, carcinoembryonic antigen (CEA), CAP-1, CAP-2, etv6, AMLI, prostate specific antigen (PSA), PSA-1, PSA-2, PSA-3, prostate-specific membrane antigen (PSMA), T-cell receptor/CD3-zeta chain, and CD₂O. The cancer antigen may be selected from the group consisting of MAGE-Al, MAGE-A2, MAGE-A3, MAGE-A4, MAGE-A5, MAGE-A6, MAGE-A7, MAGE-A8, MAGE-A9, MAGE-A10, MAGE-All, MAGE-Al2, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel}117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-I, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

Microbial Antigens. Microbial antigens are antigens derived from microbial species such as without limitation bacterial, viral, fungal, parasitic and mycobacterial species. As such, microbial antigens include bacterial antigens, viral antigens, fungal antigens, parasitic antigens, and mycobacterial antigens. Examples of bacterial, viral, fungal, parasitic and mycobacterial species are provided herein. The microbial antigen may be part of a microbial species or it may be the entire microbe.

Allergens. An allergen is an agent that can induce an allergic or asthmatic response in a subject. Allergens include without limitation pollens, insect venoms, animal dander dust, fungal spores and drugs (e.g. penicillin). Examples of natural, animal and plant allergens include but are not limited to proteins specific to the following genera: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elation); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Adjuvants. The adjuvant may be without limitation saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS1312, water-based nanoparticles combined with a soluble immunostimulant, Seppic) Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod, resiquimod). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages. In these latter instances, the adjuvant may be incorporated or be an integral part of the nucleic acid gel and will be released as the gel is degraded.

Immunoinhibitory Agents. As used herein, an immunoinhibitory agent is an agent that inhibits an immune response in a subject to whom it is administered, whether alone or in combination with another agent. Examples include steroids, retinoic acid, dexamethasone, cyclophosphamide, anti-CD3 antibody or antibody fragment, and other immunosuppressants.

Growth Factors. The nanoparticles may comprise growth factors including without limitation VEGF-A, VEGF-C P1GF, KDR, EGF, HGF, FGF, angiopoietin-1, cytokines, endothelial nitric oxide synthases eNOS and iNOS, G-CSF, GM-CSF, VEGF, aFGF, SCF (c-kit ligand), bFGF, TNF, heme oxygenase, AKT (serine-threonine kinase), HIF.alpha.(hypoxia inducible factor), Del-1 (developmental embryonic locus-1), NOS (nitric oxide synthase), BMP's (bone morphogenic proteins), SERCA2a (sarcoplasmic reticulum calcium ATPase), beta-2-adrenergic receptor, SDF-1, MCP-1, other chemokines, interleukins and combinations thereof.

Anti-Cancer Agents. As used herein, an anti-cancer agent is an agent that at least partially inhibits the development or progression of a cancer, including inhibiting in whole or in part symptoms associated with the cancer even if only for the short term. Several anti-cancer agents can be categorized as DNA damaging agents and these include topoisomerase inhibitors (e.g., etoposide, ramptothecin, topotecan, teniposide, mitoxantrone), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), anthracyclines, vinca alkaloids. or epipodophyllotoxins.

Examples of anti-cancer agents include without limitation Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Bortezomib (VELCADE); Brequinar Sodium; Bropirimine; Busulfan; Cactinomycin; Calusterone; Caracemide; Carbetimer; Carboplatin (a platinum-containing regimen); Carmustine; Carubicin Hydrochloride; Carzelesin; Cedefingol; Chlorambucil; Cirolemycin; Cisplatin (a platinum-containing regimen); Cladribine; Crisnatol Mesylate; Cyclophosphamide; Cytarabine; Dacarbazine; Dactinomycin; Daunorubicin; Decitabine; Dexormaplatin; Dezaguanine; Diaziquone; Docetaxel (TAXOTERE); Doxorubicin (DOXIL); Droloxifene; Dromostanolone; Duazomycin; Edatrexate; Eflornithine; Elsamitrucin; Enloplatin; Enpromate; Epipropidine; Epirubicin; Erbulozole; Erlotinib (TARCEVA), Esorubicin; Estramustine; Etanidazole; Etoposide; Etoprine; Fadrozole; Fazarabine; Fenretinide; Floxuridine; Fludarabine; 5-Fluorouracil; Fluorocitabine; Fosquidone; Fostriecin; Gefitinib (IRESSA), Gemcitabine; Hydroxyurea; Idarubicin; Ifosfamide; Ilmofosine; Imatinib mesylate (GLEEVAC); Interferon alpha-2a; Interferon alpha-2b; Interferon alpha-n1; Interferon alpha-n3; Interferon beta-I a; Interferon gamma-I b; Iproplatin; Irinotecan; Lanreotide; Lenalidomide (REVLIMID, REVIMID); Letrozole; Leuprolide; Liarozole; Lometrexol; Lomustine; Losoxantrone; Masoprocol; Maytansine; Mechlorethamine; Megestrol; Melengestrol; Melphalan; Menogaril; Mercaptopurine; Methotrexate; Metoprine; Meturedepa; Mitindomide; Mitocarcin; Mitocromin; Mitogillin; Mitomalcin; Mitomycin; Mitosper; Mitotane; Mitoxantrone; Mycophenolic Acid; Nocodazole; Nogalamycin; Ormaplatin; Oxisuran; Paclitaxel; Pemetrexed (ALIMTA), Pegaspargase; Peliomycin; Pentamustine; Pentomone; Peplomycin; Perfosfamide; Pipobroman; Piposulfan; Piritrexim Isethionate; Piroxantrone; Plicamycin; Plomestane; Porfimer; Porfiromycin; Prednimustine; Procarbazine; Puromycin; Pyrazofurin; Riboprine; Rogletimide; Safingol; Semustine; Simtrazene; Sitogluside; Sparfosate; Sparsomycin; Spirogermanium; Spiromustine; Spiroplatin; Streptonigrin; Streptozocin; Sulofenur; Talisomycin; Tamsulosin; Taxol; Taxotere; Tecogalan; Tegafur; Teloxantrone; Temoporfin; Temozolomide (TEMODAR); Teniposide; Teroxirone; Testolactone; Thalidomide (THALOMID) and derivatives thereof; Thiamiprine; Thioguanine; Thiotepa; Tiazofurin; Tirapazamine; Topotecan; Toremifene; Trestolone; Triciribine; Trimetrexate; Triptorelin; Tubulozole; Uracil Mustard; Uredepa; Vapreotide; Verteporfin; Vinblastine; Vincristine; Vindesine; Vinepidine; Vinglycinate; Vinleurosine; Vinorelbine; Vinrosidine; Vinzolidine; Vorozole; Zeniplatin; Zinostatin; Zorubicin.

The anti-cancer agent may be an enzyme inhibitor including without limitation tyrosine kinase inhibitor, a CDK inhibitor, a MAP kinase inhibitor, or an EGFR inhibitor. The tyrosine kinase inhibitor may be without limitation Genistein (4′,5,7 trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, or HDBA (2-Hydroxy-5-(2,5-Dihydroxybenzylamino)-2-hydroxybenzoic acid. The CDK inhibitor may be without limitation p21, p27, p57, p15, p16, p18, or p19. The MAP kinase inhibitor may be without limitation KY12420 (C₂₃H₂₄O₈), CNI-1493, PD98059, or 4-(4-Fluorophenyl)-2-(4-methylsulfinyl phenyl)-5-(4-pyridyl) 1H-imidazole. The EGFR inhibitor may be without limitation erlotinib (TARCEVA), gefitinib (IRESSA), WHI-P97 (quinazoline derivative), LFM-A12 (leflunomide metabolite analog), ABX-EGF, lapatinib, canertinib, ZD-6474 (ZACTIMA), AEE788, and AG1458.

The anti-cancer agent may be a VEGF inhibitor including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia,), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma R1)), oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), IOR-05, IOR-T6 (anti-CD1), IOR EGF/R3, celogovab (ONCOSCINT OV103), epratuzumab (LYMPHOCIDE), pemtumomab (THERAGYN), and Gliomab-H (indicated for brain cancer, melanoma).

Anti-Infective Agents. The agent may be an anti-infective agent including without limitation an anti-bacterial agent, an anti-viral agent, an anti-parasitic agent, an anti-fungal agent, and an anti-mycobacterial agent.

Anti-bacterial agents may be without limitation β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, or quinolines.

Other anti-bacterials may be without limitation Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin.

Anti-mycobacterial agents may be without limitation Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate or Trecator-SC (Ethionamide).

Anti-viral agents may be without limitation amantidine and rimantadine, ribivarin, acyclovir, vidarabine, trifluorothymidine, ganciclovir, zidovudine, retinovir, and interferons.

Anti-viral agents may be without limitation further include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime or integrase inhibitors.

Anti-fungal agents may be without limitation imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhibitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); or sulfated naphthylamine (e.g., suramin).

Other anti-infective agents may be without limitation Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; or Troclosene Potassium.

Other Agents. The agent may be without limitation adrenergic agent; adrenocortical steroid; adrenocortical suppressant; alcohol deterrent; aldosterone antagonist; ammonia detoxicant; amino acid; amylotropic lateral sclerosis agent; anabolic; analeptic; analgesic; androgen; anesthetic; anorectic; anorexic; anterior pituitary activator; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic including β-adrenergic agonists, methylxanthines, mast cell stabilizing agents, anticholinergics, adrenocortical steroids such as glucocorticoids; anti-atherosclerotic; anticholelithic; anticholelithogenic; anticholinergic; anticoagulant; anticoccidal; anticonvulsant; antidepressant; antidiabetic; antidiarrheal; antidiuretic; antidote; antidyskinetic; anti-emetic; anti-epileptic; anti-estrogen; antifibrinolytic; antiglaucoma; antihemorrhagic; antihemorrheologic; antihistamine; antihyperlipidemic; antihyperlipoproteinemic; antihypertensive; antihypotensive; anti-infective; anti-inflammatory; antikeratinizing agent; antimigraine; antimitotic; antimycotic; antinauseant; antineutropenic; antiobsessional agent; antioxidant; antiparkinsonian; antiperistaltic; antipneumocystic; antiprostatic hypertrophy agent; antiprotozoal; antipruritic; antipsoriatic; antipsychotic; antirheumatic; antischistosomal; antiseborrheic; antisecretory; antispasmodic; antithrombotic; antitussive; anti-ulcerative; anti-urolithic; appetite suppressant; blood glucose regulator; bone resorption inhibitor; bronchodilator; carbonic anhydrase inhibitor; cardiac depressant; cardioprotectant; cardiotonic; cardiovascular agent; cerebral ischemia agent; choleretic; cholinergic; cholinergic agonist; cholinesterase deactivator; coccidiostat; cognition adjuvant; cognition enhancer; conjunctivitis agent; contrast agent; depressant; diagnostic aid; diuretic; dopaminergic agent; ectoparasiticide; emetic; enzyme inhibitor; estrogen; estrogen receptor agonist; fibrinolytic; fluorescent agent; free oxygen radical scavenger; gastric acid suppressant; gastrointestinal motility effector; geriatric agent; glucocorticoid; gonad-stimulating principle; hair growth stimulant; hemostatic; herbal active agent; histamine H2 receptor antagonists; hormone; hypocholesterolemic; hypoglycemic; hypolipidemic; hypotensive; HMGCoA reductase inhibitor; impotence therapy adjunct; inflammatory bowel disease agent; keratolytic; LHRH agonist; liver disorder agent; luteolysin; memory adjuvant; mental performance enhancer; mineral; mood regulator; mucolytic; mucosal protective agent; multiple sclerosis agent; mydriatic; nasal decongestant; neuroleptic; neuromuscular blocking agent; neuroprotective; NMDA antagonist; non-hormonal sterol derivative; nutrient; oxytocic; Paget's disease agent; plasminogen activator; platelet activating factor antagonist; platelet aggregation inhibitor; post-stroke and post-head trauma agents; progestin; prostaglandin; prostate growth inhibitor; prothyrotropin; psychotropic; radioactive agent; relaxant; rhinitis agent; scabicide; sclerosing agent; sedative; sedative-hypnotic; selective adenosine A1 antagonist; sequestering agents; serotonin antagonist; serotonin inhibitor; serotonin receptor antagonist; steroid; stimulant; suppressant; thyroid hormone; thyroid inhibitor; thyromimetic; tranquilizer; unstable angina agent; uricosuric; vasoconstrictor; vasodilator; vulnerary; wound healing agent; or xanthine oxidase inhibitor.

### In Vitro Use

The invention further contemplates in vitro applications such as cell culturing and tissue engineering, that require or for which it would be more convenient to have a constant source of one or more agents such as but not limited to cell growth factors, and the like.

### Subjects

When the monomers, hydrogels and/or nanoparticles are used in vivo, the invention can be practiced in virtually any subject type that is likely to benefit prophylactically, therapeutically, or prognostically from the delivery of siRNA and optionally one or more other agents as contemplated herein.

Human subjects are preferred subjects in some embodiments of the invention. Subjects also include animals such as household pets (e.g., dogs, cats, rabbits, ferrets, etc.), livestock or farm animals (e.g., cows, pigs, sheep, chickens and other poultry), horses such as thoroughbred horses, laboratory animals (e.g., mice, rats, rabbits, etc.), and the like. Subjects also include fish and other aquatic species.

The subjects to whom the agents are delivered may be normal subjects. Alternatively they may have or may be at risk of developing a condition that can be diagnosed or that can benefit or that can be prevented from systemic or localized delivery of siRNA and optionally one or more other agents. Such conditions include cancer (e.g., solid tumor cancers), infections (particularly infections localized to particular regions or tissues in the body), autoimmune disorders, allergies or allergic conditions, asthma, transplant rejection, diabetes, heart disease, and the like.

Tests for diagnosing various of the conditions embraced by the invention are known in the art and will be familiar to the ordinary medical practitioner. These laboratory tests include without limitation microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner will generally also take a full history and conduct a complete physical examination in addition to running the laboratory tests listed above.

A subject having a cancer is a subject that has detectable cancer cells. A subject at risk of developing a cancer is a subject that has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

Subjects having an infection are those that exhibit symptoms thereof including without limitation fever, chills, myalgia, photophobia, pharyngitis, acute lymphadenopathy, splenomegaly, gastrointestinal upset, leukocytosis or leukopenia, and/or those in whom infectious pathogens or byproducts thereof can be detected.

A subject at risk of developing an infection is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery, etc.

The subject may have or may be at risk of developing an infection such as a bacterial infection, a viral infection, a fungal infection, a parasitic infection or a mycobacterial infection. In these embodiments, the nanoparticles may comprise an anti-microbial agent such as an anti-bacterial agent, an anti-viral agent, an anti-fungal agent, an anti-parasitic agent, or an anti-mycobacterial agent and the cell carriers (e.g., the T cells) may be genetically engineered to produce another agent useful in stimulating an immune response against the infection, or potentially treating the infection.

### Cancer

The invention contemplates administration of monomers, hydrogels and/or nanoparticles to subjects having or at risk of developing a cancer including for example a solid tumor cancer. The cancer may be carcinoma, sarcoma or melanoma. Carcinomas include without limitation to basal cell carcinoma, biliary tract cancer, bladder cancer, breast cancer, cervical cancer, choriocarcinoma, CNS cancer, colon and rectum cancer, kidney or renal cell cancer, larynx cancer, liver cancer, small cell lung cancer, non-small cell lung cancer (NSCLC, including adenocarcinoma, giant (or oat) cell carcinoma, and squamous cell carcinoma), oral cavity cancer, ovarian cancer, pancreatic cancer, prostate cancer, skin cancer (including basal cell cancer and squamous cell cancer), stomach cancer, testicular cancer, thyroid cancer, uterine cancer, rectal cancer, cancer of the respiratory system, and cancer of the urinary system.

Sarcomas are rare mesenchymal neoplasms that arise in bone (osteosarcomas) and soft tissues (fibrosarcomas). Sarcomas include without limitation liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., not bone) Ewing's sarcoma, and primitive neuroectodermal tumor), synovial sarcoma, angiosarcomas, hemangiosarcomas, lymphangiosarcomas, Kaposi's sarcoma, hemangioendothelioma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), and chondrosarcoma.

Melanomas are tumors arising from the melanocytic system of the skin and other organs. Examples of melanoma include without limitation lentigo maligna melanoma, superficial spreading melanoma, nodular melanoma, and acral lentiginous melanoma.

The cancer may be a solid tumor lymphoma. Examples include Hodgkin's lymphoma, Non-Hodgkin's lymphoma, and B cell lymphoma.

The cancer may be without limitation bone cancer, brain cancer, breast cancer, colorectal cancer, connective tissue cancer, cancer of the digestive system, endometrial cancer, esophageal cancer, eye cancer, cancer of the head and neck, gastric cancer, intra-epithelial neoplasm, melanoma neuroblastoma, Non-Hodgkin's lymphoma, non-small cell lung cancer, prostate cancer, retinoblastoma, or rhabdomyosarcoma.

### Infection

The invention contemplates administration of monomers, hydrogels and/or nanoparticles to subjects having or at risk of developing an infection such as a bacterial infection, a viral infection, a fungal infection, a parasitic infection or a mycobacterial infection.

The bacterial infection may be without limitation an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteriodes infection, H. pylori infection, or anthrax infection.

The mycobacterial infection may be without limitation tuberculosis or leprosy respectively caused by the M. tuberculosis and M. leprae species.

The viral infection may be without limitation a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection, SARS infection or avian flu infection.

The fungal infection may be without limitation candidiasis, ringworm, histoplasmosis, blastomycosis, paracoccidioidomycosis, crytococcosis, aspergillosis, chromomycosis, mycetoma infections, pseudallescheriasis, or tinea versicolor infection.

The parasite infection may be without limitation amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginalis infection, Taenia infection, Hymenolepsis infection, Echinococcus infections, Schistosomiasis, neurocysticercosis, Necator americanus infection, or Trichuris trichuria infection.

### Allergy and Asthma

The invention contemplates administration of monomers, hydrogels and/or nanoparticles to subjects having or at risk of developing an allergy or asthma. An allergy is an acquired hypersensitivity to an allergen. Allergic conditions include but are not limited to eczema, allergic rhinitis or coryza, hay fever, bronchial asthma, urticaria (hives) and food allergies, and other atopic conditions. Allergies are generally caused by IgE antibody generation against harmless allergens. Asthma is a disorder of the respiratory system characterized by inflammation, narrowing of the airways and increased reactivity of the airways to inhaled agents. Asthma is frequently, although not exclusively, associated with atopic or allergic symptoms. Administration of Th1 cytokines, such as IL-12 and IFN-gamma, according to the invention can be used to treat allergy or asthma.

### Autoimmune Disease

The invention contemplates administration of monomers, hydrogels and/or nanoparticles to subjects having or at risk of developing an autoimmune disease.

Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases are generally considered to be Th1 biased. As a result, induction of a Th2 immune response or Th2 like cytokines can be beneficial. Such cytokines include IL-4, IL-5 and IL-10.

Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

### Transplant Therapy

The monomers, hydrogels and/or nanoparticles provided herein may also be used to modulate immune responses following transplant therapy. Transplant success is often limited by rejection of the transplanted tissue by the body's immune system. As a result, transplant recipients are usually immunosuppressed for extended periods of time in order to allow the transplanted tissue to survive. The invention contemplates localized (e.g., to transplant sites, organs or tissues) or in some instances systemic delivery of immunomodulators, and particularly immunoinhibitory agents, in order to minimize transplant rejection. Thus, the invention contemplates administration of the nanoparticles to subjects that are going to undergo, are undergoing, or have undergone a transplant.

The foregoing lists are not intended to be exhaustive but rather exemplary. Those of ordinary skill in the art will identify other examples of each condition type that are amenable to prevention and treatment using the methods of the invention.

### Effective Amounts, Regimens, Formulations

The agents are administered in effective amounts. An effective amount is a dosage of the agent sufficient to provide a medically desirable result. The effective amount will vary with the particular condition being treated, the age and physical condition of the subject being treated, the severity of the condition, the duration of the treatment, the nature of the concurrent or combination therapy (if any), the specific route of administration and like factors within the knowledge and expertise of the health practitioner. It is preferred generally that a maximum dose be used, that is, the highest safe dose according to sound medical judgment.

For example, if the subject has a tumor, an effective amount may be that amount that reduces the tumor volume or load (as for example determined by imaging the tumor). Effective amounts may also be assessed by the presence and/or frequency of cancer cells in the blood or other body fluid or tissue (e.g., a biopsy). If the tumor is impacting the normal functioning of a tissue or organ, then the effective amount may be assessed by measuring the normal functioning of the tissue or organ.

Administration may be a systemic route such as intravenous, intramuscular, intradermal, intraperitoneal, subcutaneous, by inhalation, or other parenteral routes. Administration may be oral or it may be through a localized route such as injection or topical administration to a tissue (e.g., skin, mucosa such as oral, vaginal, rectal, gut, or lung mucosa), an organ, a tumor, a lesion, a site of infection such as an abscess, and the like. The route of administration in some instances will be governed by the particular condition being treated or diagnosed.

The invention provides pharmaceutical compositions. Pharmaceutical compositions are sterile compositions that comprise nanoparticles and embedded agent(s), preferably in a pharmaceutically-acceptable carrier. The term “pharmaceutically-acceptable carrier” means one or more compatible solid or liquid filler, diluents or encapsulating substances which are suitable for administration to a human or other subject contemplated by the invention. The term “carrier” denotes an organic or inorganic ingredient, natural or synthetic, with which the cells, nanoparticles and agent(s) are combined to facilitate administration. The components of the pharmaceutical compositions are comingled in a manner that precludes interaction that would substantially impair their desired pharmaceutical efficiency.

When delivered systemically, the monomers, hydrogels and/or nanoparticles may be formulated for parenteral administration by injection, e.g., by bolus injection or continuous infusion. Formulations for injection may be presented in unit dosage form, e.g., in ampoules or in multi-dose containers. Pharmaceutical parenteral formulations include aqueous solutions of the ingredients. Aqueous injection suspensions may contain substances which increase the viscosity of the suspension, such as sodium carboxymethyl cellulose, sorbitol, or dextran. Alternatively, suspensions of ingredients may be prepared as oil-based suspensions. Suitable lipophilic solvents or vehicles include fatty oils such as sesame oil, or synthetic fatty acid esters, such as ethyl oleate or triglycerides.

The following Examples are included for purposes of illustration and are not intended to limit the scope of the invention.

### EXAMPLES

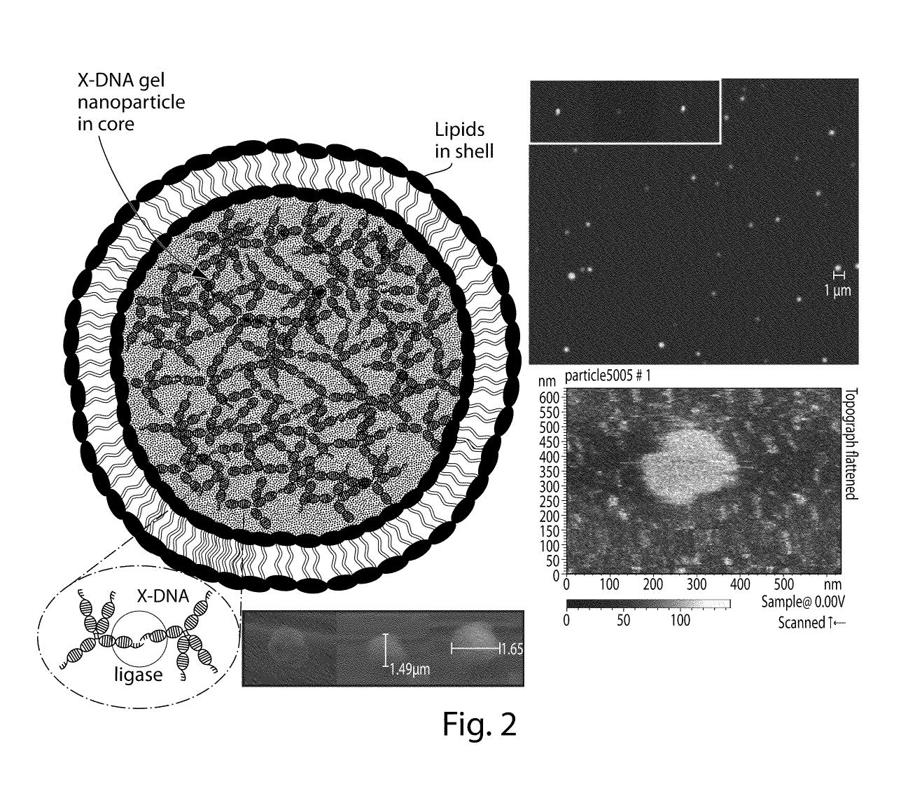

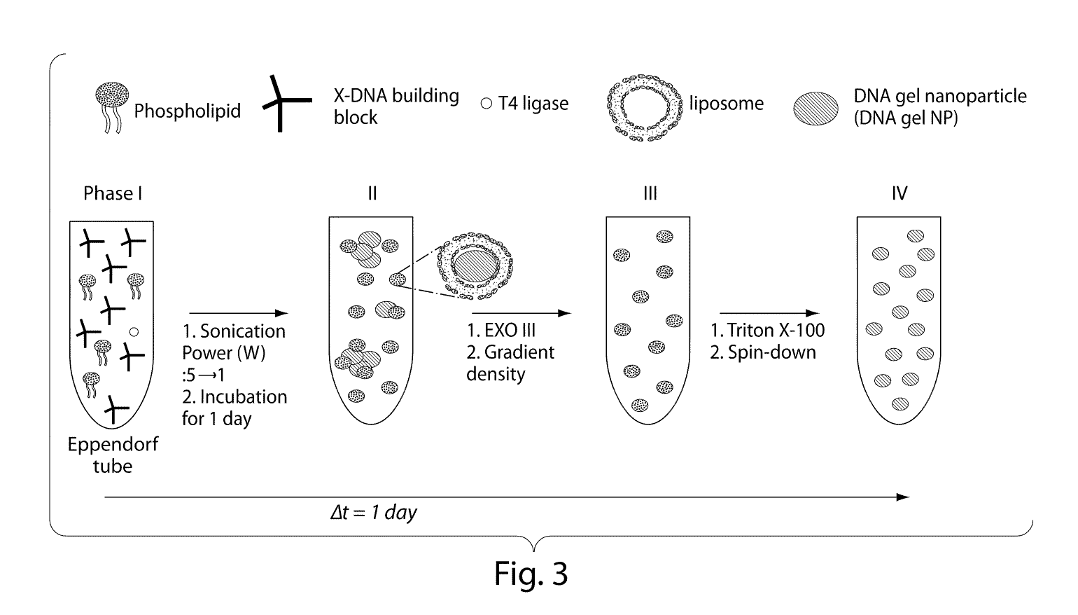

Synthesis of DNA nanogels with or without a lipid coating. The overall structure of exemplary DNA nanogels and an exemplary synthetic process (using “X-DNA” monomers) for their production is outlined in FIGS. 2 and 3, respectively. As summarized in FIG. 3, the nanogels are synthesized by a simple multistep process. First, X-DNA monomers (or building blocks), composed of 4 individual DNA strands designed to hybridize with one another into a characteristic 4-armed structure are prepared using standard molecular biology techniques. See also published US patent applications US 20070148246 A1 and US 20050130180 A1. These DNA building blocks are then encapsulated into liposomes by rehydrating a dried phospholipid film in a vial with an aqueous solution of X-DNA and the crosslinking enzyme T4 ligase, and sonicating the lipid/DNA/enzyme mixture briefly. The size of the liposomes formed establishes the size of the resulting DNA nanogels. These liposome-like entities may then be size selected for example by passing them through membranes of reducing pore size. In this manner, populations of nanogels with a common average size can be generated. The mixture may be treated to remove free, unencapsulated nucleic acid before or after size separation and before or after crosslinking of the encapsulated nucleic acids, as discussed below.

Before or after size selection, the nanogels are incubated to covalently crosslink the ends of adjacent X-DNA arms to one another. If the crosslinking agent is T4 ligase, then a suitable incubation is 24 hrs at 16° C. (or room temperature). Other incubation times and conditions may be used, as will be apparent to those of ordinary skill in the art in accordance with the teachings herein. The resultant reaction mixture comprises crosslinked DNA gels encapsulated by lipid coatings (or liposomes) as well as “free” crosslinked DNA gel which is formed and exists outside of the liposomes (FIG. 3 Step II). Because this free DNA gel forms without a lipid “template” it does not adopt a nanogel form and instead is much larger.

Free unencapsulated DNA gel then may be degraded by treating the mixture with nuclease(s) such as exonuclease(s). The nuclease(s) targets and degrades only the unencapsulated DNA, whether or not crosslinked, while the encapsulated DNA remains intact. The mixture is finally purified by centrifugation through a sucrose density gradient to remove DNA fragments and free liposomes (FIG. 3 Step III). If lipid-free (or “naked”) DNA nanogels are desired, the purified DNA nanogels are treated to remove their lipid coating in a final step (FIG. 3 Step IV). Lipid coats may be removed using detergent such as Triton-X-100 or enzymes such as lipases and phospholipases.

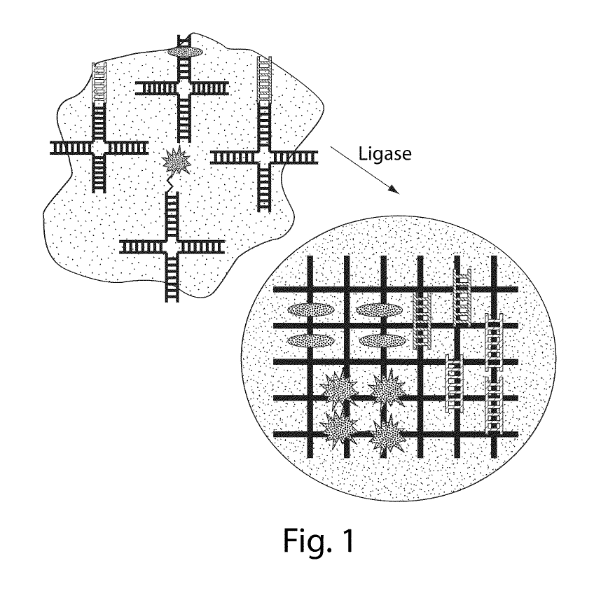

FIGS. 1 and 2 schematically illustrate the final structure of DNA nanogels formed by crosslinking X-DNA monomers. X-DNA monomers are crosslinked arm-to-arm to form a 3D network within liposomal vesicles. Nanogels with sizes from ˜1 μm down to ˜100 nm diameter can be synthesized by changing the concentration of reactants and the types of lipids used in the synthesis. Also shown in FIG. 2 are confocal micrographs and a fluid-cell AFM image of DNA nanogels formed with this process. Nanogels with a range of net sizes and surface charge can be prepared with a variety of lipid coating compositions (Table 2).

TABLE 2
DNA nanoparticles with a variety of lipid components

| Sample Description | | Zeta- | Product |
|---|---|---|---|
| (e.g., DNA gel nanoparticle with the | Size | Potential | Yield |
| lipid components: DOPC X%/DOPG Y%) | (nm)* | (mV) | (%) |
| DOPC 90%/Rhod-DOPC 10% | 857 ± 28 | 0.652 | 52.0 |
| DNA nanogel alone (after lipid extraction) | 857 ± 28 | −29.04 | 48.0 |
| DOPC 90%/PEG-DSPE 10% | 100.6 ± 0.7 | 0.00147 | (very low) |
| DOPC 72%/DOPG 18%/PEG-DSPE 10% | 304.8 ± 13.2 | −3.55 | 21.6 |
| DOPC 40%/DOPG 10%/MBP-PE 50% | 797.0 ± 52.7 | 0.243 | 54.0 |

Sized by 1 micron membrane extrusion prior to crosslinking

| | | | |
|---|---|---|---|
| DOPC | 445.9 ± 24.4 | 0.223 | 58.0 |
| DOPC 40%/DOPG 10%/MBP-DOPE 50% | 310.2 ± 12.4 | −0.0268 | 52.0 |

Sized by 400 nm membrane extrusion prior to crosslinking

| | | | |
|---|---|---|---|
| DOPC 40%/DOPG 10%/MCC-DOPE 50% | 334.2 ± 4.8 | 5.39 | 48.0 |

Sized by 200 nm membrane extrusion prior to crosslinking

| | | | |
|---|---|---|---|
| DOPC 40%/DOPG 10%/MBP-DOPE 50% | 258.6 ± 6.8 | −0.027 | 54.0 |

*determined by dynamic light scattering

Lipid compositions compatible with DNA nanogel synthesis. The synthesis steps described above represent an example of an optimized synthesis scheme. It has been found according to the invention that not all lipid types can be used to prepare well-defined submicron DNA nanogels. As shown in Table 2, nanogels readily formed when zwitterionic (DOPC) and/or anionic (DOPG) phospholipids were used in the synthesis. However, addition of lipids (e.g., DSPE) conjugated to polyethylene glycol (PEG) (e.g., PEG-DSPE) reduced the yield of DNA nanogels (Table 2). Moreover, when cationic phospholipids such as DOTAP were employed in the synthesis, macroscopic DNA-lipid aggregates formed and the yield of nanogels was also very low. Thus, neutral and/or anionic lipid compositions lacking PEG headgroups appear to be optimal for synthesis of submicron DNA nanogels. If PEGylation is desired, however, it has also been determined in accordance with the invention that lipid-coated DNA nanogels are readily PEGylated post-synthesis, for example by reacting thiol-terminated PEG with maleimide-functionalized lipids used to generate the nanogels in the first instance.

It has also been found that nanogel formation preferably occurs under certain molar ratios of X-DNA:lipid. As shown in FIG. 4 (left panel), the mean size of DNA nanogels formed in this synthesis varies with the lipid:X-DNA mole ratio (nₗ/n_{d}), with the mean particle radius (and thus also diameter) roughly inversely proportional to this ratio. Lipid:X-DNA ratios near ˜10 are suitable for generating submicron-sized nanogels. At lower ratios, macroscopic DNA-gel aggregates are formed (FIG. 4, right panel).

Design of DNA-RNA hybrid X′ nanostructures for gene silencing with siRNA. FIG. 3 illustrates a synthetic approach to prepare crosslinked hydrogel nanoparticles composed of for example a crosslinked double-stranded DNA network. These particles can be prepared with or without a liposomal shell. These DNA-based nanoparticles were able to stably encapsulate high levels of the chemotherapy drug doxorubicin or globular proteins, which can be released in a slow and sustained manner up to 1 month.

In order to extend these findings, we first tested different DNA-RNA hybrid structures to find compositions of X-DNA molecules that could carry siRNA arms competent for gene silencing. As shown in FIG. 4, we found the ‘L’-form siRNA was weakly functional in gene silencing in vitro when linked as an arm of X-DNAs, while ‘R’ form siRNA added as arms of the X-DNA structures was nearly as potent as free siRNA in silencing luciferase expression in 293T cells.

siRNA/DNA X′ nanostructures and siRNA/DNA-nanogels can potently silence stably transfected genes in tumor cells and achieve more prolonged silencing than free siRNA.

Having established an ‘X’ DNA-siRNA hybrid structure capable of silencing genes in this co-transfection experiment, we next tested the silencing of a stably expressed gene in B16F10 melanoma cells. B16 cells expressing green fluorescent protein (GFP) were treated with siRNA-DNA ‘X’ nanostructures bearing 1, 2, 3, or 4 siRNA arms, or DNA-nanogels prepared with siRNA-DNA molecules bearing 1 siRNA arm on each ‘X’ molecule in the presence of a commercial lipid transfection reagent to promote cytosolic delivery of the hybrid siRNA molecules. As shown in FIG. 5, X-DNA structures with 2, 3, or 4 siRNA arms were effective in silencing GFP in the tumor cells, and were in fact somewhat more efficient in silencing this stably expressed gene than standard free ‘R’-form siRNA. Further, X-DNA-nanogels prepared containing siRNA were equally potent in silencing GFP expression. Interestingly, when we titrated the total dose of siRNA applied to B16 cells, both ‘X’ siRNA/DNA nanostructures and siRNA/DNA-nanogels silenced more effectively at low total siRNA doses than free siRNA (FIG. 5, right panel).

Because siRNA/DNA-nanogels contain siRNA duplexes throughout the gel network, we hypothesized that fresh siRNA molecules would be continuously released over time as the network is degraded in cells, paralleling the slow DNA release seen in our in vitro dox release studies. To determine if this is the case, we silenced GFP in B16-GFP tumor cells with free siRNA or siRNA/DNA-nanogels, and measured GFP expression as a function of time in the B16 cells. As shown in FIG. 6, free siRNA and nanogels were equivalently effective in knocking down GFP expression over the first 24 hrs post transfection, but nanogels continued to suppress GFP expression for 3 days, while free siRNA silencing was completely recovered by 48 hrs. Thus siRNA/DNA hybrid nanogels offer the possibility of sustained gene silencing, which could be of great interest for in vivo therapeutic applications.

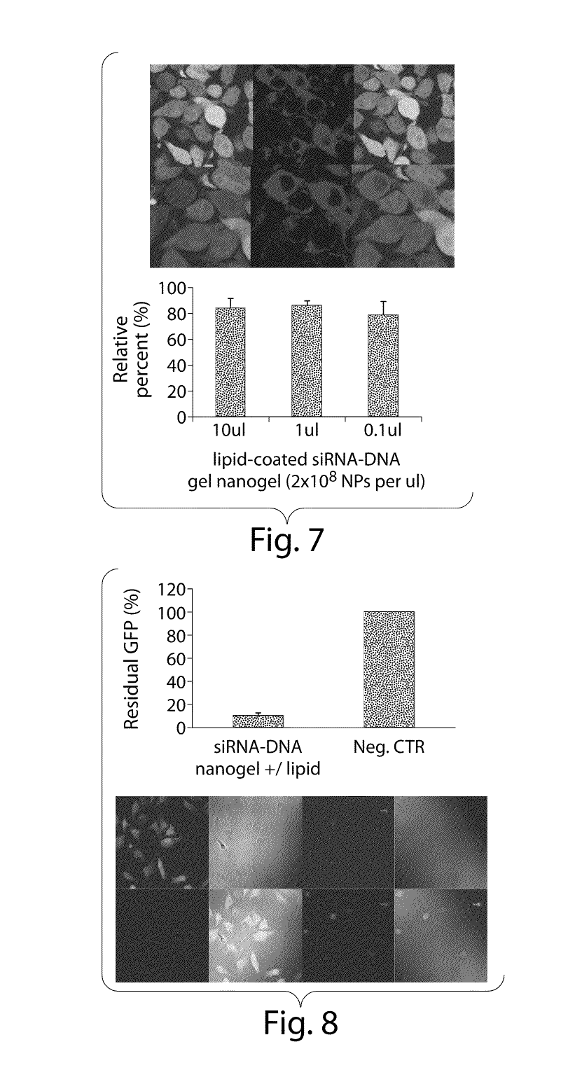

Lipid-coated DNA-nanogels are avidly internalized by tumor cells, are nontoxic, and elicit strong gene silencing. Finally, for in vivo applications, the ideal system would combine this siRNA/DNA-nanogel hybrid structure with components promoting efficient cytosolic delivery of the particles, replacing the commercial cationic lipid transfection reagent used in our in vitro silencing experiments which is likely toxic in vivo. Recently, it has been reported that neutral zwitterionic liposomes can deliver siRNA systemically in vivo to tumors following i.p. injection^{1,2}. We thus examined the uptake of zwitterionic lipid-coated DNA-nanogels, with a liposomal shell containing a 4:1 mol:mol ratio of the zwitterionic lipid DOPC and anionic lipid DOPG. As shown in FIG. 7, these lipid-coated DNA-nanogels are very efficiently internalized by B16-GFP melanoma tumors cells, but do not cause toxicity on their own. Importantly, B16F0 melanoma cells stably expressing GFP that were treated with lipid-coated DNA/siRNA-nanogels encoding a GFP-directed siRNA promoted strong knockdown of GFP fluorescence in B16FO cells in vitro (FIG. 8). Thus, we now have a nontoxic, noncationic siRNA delivery system that can carry high payloads of siRNA within nanoparticles, is fully biodegradable, and can achieve gene knockdown in vitro comparable to free standard siRNA transfection methods. We are currently preparing tests of in vivo gene silencing to further extend these findings.

### REFERENCES

1. Landen, C. N., Jr. et al. Therapeutic EphA2 gene targeting in vivo using neutral liposomal small interfering RNA delivery. Cancer Res 65, 6910-8 (2005).
2. Villares, G. J. et al. Targeting melanoma growth and metastasis with systemic delivery of liposome-incorporated protease-activated receptor-1 small interfering RNA. Cancer Res 68, 9078-86 (2008).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A complex comprising
a branched nucleic acid, and
an R-form siRNA linked to the branched nucleic acid.

2-8. (canceled)

9. A hydrogel comprising the complex of claim 1, wherein the complex is crosslinked.

10-31. (canceled)

32. A method comprising
administering the complex of claim 1 or the hydrogel of claim 9 to a subject in need thereof in an effective amount.

33. A method comprising
reducing expression of a target protein in a cell by contacting the cell with a complex of claim 1 or a hydrogel of claim 9, wherein the complex or hydrogel comprises a target-specific siRNA.

34. A method comprising
reducing expression of a target protein in vivo for a period of 2-3 days following administration to a subject of a complex of claim 1 or a hydrogel of claim 9, wherein the complex or hydrogel comprises a target-specific siRNA.

35-46. (canceled)

47. A hydrogel produced by a method comprising crosslinking branched nucleic acids attached to R-form siRNA in the presence of a DNA ligase.

48. A composition comprising
a hydrogel comprising crosslinked branched DNA complexes attached to R-form siRNA.

49-64. (canceled)

65. A method comprising
introducing a hydrogel of claim 47 to a subject in need thereof in an effective amount.

66-72. (canceled)

73. A method comprising
reducing expression of a target protein in vivo for a period of 2-3 days following administration to a subject of a DNA hydrogel that comprises target-specific siRNA.

74.-160. (canceled)

161. The complex of claim 1, wherein the branched nucleic acid is an X-shaped nucleic acid.

162. The complex of claim 1, wherein the branched nucleic acid is a Y-shaped nucleic acid.

163. The complex of claim 1, wherein the branched nucleic acid is a branched DNA.

164. The complex of claim 1, wherein the R-form siRNA is covalently linked to the branched nucleic acid.

165. The complex of claim 1, wherein the R-form siRNA is non-covalently linked to the branched nucleic acid.

166. The complex of claim 1, wherein the branched nucleic acid is linked to 2, 3 or 4 siRNA.

167. The complex of claim 1, wherein an R-form siRNA is linked to each arm of the branched nucleic acid.

168. The hydrogel of claim 9, wherein the complexes are a heterogeneous mixture of complexes.

169. The hydrogel of claim 9, wherein the complexes are a homogeneous mixture of complexes.
